(19) United States
(12) Patent Application Publication
(10) Pub. No.: US 2025/0032440 A1
(43) Pub. Date: Jan. 30, 2025

(54) COMPOSITIONS AND METHODS FOR TREATING NEURODEGENERATIVE DISORDERS

(71) Applicant: ARIZONA BOARD OF REGENTS ON BEHALF OF THE UNIVERSITY OF ARIZONA, Tucson, AR (US)

(72) Inventors: Rui CHANG, Tucson, AZ (US); Wei Wang, Tucson, AZ (US); Patrick T. Ronaldson, Tucson, AZ (US); Qianying He, Tucson, AZ (US); Yanyun Liu, Tucson, AZ (US); Ramachandran Vijayan, Tucson, AZ (US)

(21) Appl. No.: 18/686,950

(22) PCT Filed: Aug. 25, 2022
(86) PCT No.: PCT/US2022/041565

§ 371 Date: Feb. 27, 2024

Related U.S. Application Data
(60) Provisional application No. 63/237,928, filed on Aug. 27, 2021.

(51) Int. Cl. A61K 31/26; A61K 31/404; A61K 31/4409; A61K 45/06

(52) U.S. Cl. CPC A61K 31/26; A61K 31/404; A61K 31/4409; A61K 45/06

(57) ABSTRACT

This invention relates generally to neurodegenerative diseases and conditions (e.g., Alzheimer's disease) characterized with dysfunctional energetic function, unregulated microglia phagocytic activity and other related de-regulated biological functions. This invention further relates to methods and compositions for treating such neurodegenerative diseases and conditions with pharmaceutical compositions comprising one or more of agents capable of protecting neurons from cell death and unregulated microglia phagocytic activity.

### CROSS-REFERENCE TO RELATED APPLICATIONS

This application is a U.S. National Entry of PCT/US2022/041565 filed Aug. 25, 2022, which claims priority to U.S. Provisional Application No. 63/237,928, filed Aug. 27, 2021, the entire contents of which are incorporated herein by reference for all purposes.

### STATEMENT REGARDING FEDERALLY SPONSORED RESEARCH OR DEVELOPMENT

This invention was made with government support under Grant No. R56 AG062620 and Grant No. AG057457 awarded by National Institutes of Health. The government has certain rights in the invention.

### FIELD OF THE INVENTION

This invention relates generally to neurodegenerative diseases and conditions (e.g., Alzheimer's disease) characterized with dysfunctional energetic function, unregulated microglia phagocytic activity and other related de-regulated biological functions. This invention further relates to methods and compositions for treating such neurodegenerative diseases and conditions with pharmaceutical compositions comprising one or more of agents capable of protecting neurons from cell death and unregulated microglia phagocytic activity.

### BACKGROUND OF THE INVENTION

There is an urgent need to develop novel therapies for neurodegenerative diseases and conditions such as Alzheimer's disease (AD). 10% of persons over age 65 and up to 50% over age 85 have dementia, with over 30 million people affected worldwide. AD affects over 26 million people worldwide and currently there is no cure for the disease. With the growing number of people living to older ages, there is an urgency to better understand elements of the pathogenic pathway, discover agents that target these elements, and establish their roles in the treatment and prevention of AD.

As such, improved methods for treating neurodegenerative disorders (e.g., AD) are needed.

The present invention addresses this need.

### SUMMARY

AD is a neurodegenerative disease with no effective treatment. AD is characterized by the accumulation of amyloid beta (Aβ) peptide into toxic plaques around neurons and tau protein tangles within neurons, resulting in neuronal death and overall damage to the brain. Microglia are cells found in the central nervous system (CNS) whose functions include clearing Amyloid-beta (Aβ) deposition in the AD brain via phagocytosis. Many AD drug developments have only focused on targeting and neutralizing Aβ and tau from the brain and have had little success in treating the disease as a result.

Accordingly, the present invention relates generally to neurodegenerative diseases and conditions (e.g., Alzheimer's disease) characterized with dysfunctional energetic function, unregulated microglia phagocytic activity and other related de-regulated biological functions. This invention further relates to methods and compositions for treating such neurodegenerative diseases and conditions with pharmaceutical compositions comprising one or more agents capable of protecting neurons from cell death and unregulated microglia phagocytic activity.

In certain embodiments, the present invention provides a method of treating a mammal suffering from a neurodegenerative disorder comprising administering to the mammal a composition comprising one or more agents capable of protecting neurons from cell death and unregulated microglia phagocytic activity.

In certain embodiments, the present invention provides a method for preventing and/or inhibiting neuronal cell death in a mammal in need thereof, the method comprising administering to the mammal a composition comprising one or more agents capable of protecting neurons from cell death and unregulated microglia phagocytic activity.

In certain embodiments, the present invention provides a method for preventing and/or inhibiting unregulated microglia phagocytic activity in a mammal in need thereof, the method comprising administering to the mammal a composition comprising one or more agents capable of protecting neurons from cell death and unregulated microglia phagocytic activity.

In some embodiments, the neurodegenerative disorder is selected from AD, Parkinson's disease, Huntington's disease, amyotrophic lateral sclerosis, motor neuron disease, subjective memory complaints, and mild cognitive impairment (MCI). In some embodiments, the AD is an early stage, prodromal phase of AD or late stage.

In some embodiments, the mammal is a human patient.

In certain embodiments, the present invention provides a method for preventing and/or inhibiting neuronal cell death in a subject suffering from a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) comprising, consisting of, or consisting essentially of administering to the subject a therapeutically effective amount of one or more agents capable of preventing and/or inhibiting unregulated microglia phagocytic activity.

In certain embodiments, the present invention provides for preventing and/or inhibiting unrelated microglia phagocytic activity in neuronal cells of a subject suffering from a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) comprising, consisting of, or consisting essentially of administering to the subject a therapeutically effective amount of one or more agents capable of preventing and/or inhibiting unregulated microglia phagocytic activity.

In certain embodiments, the present invention provides a method of preventing the onset of a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) in a subject (e.g., a human subject) comprising, consisting of, or consisting essentially of administering to the subject a therapeutically effective amount of one or more agents capable of preventing and/or inhibiting unregulated microglia phagocytic activity.

In certain embodiments, the present invention provides a method of treating and/or ameliorating the symptoms of a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) in a subject (e.g., a human subject) comprising, consisting of, or consisting essentially of administering to the subject a therapeutically effective amount of one or more agents capable of preventing and/or inhibiting unregulated microglia phagocytic activity.

Such methods are not limited to use of a particular agent capable of protecting neurons from cell death and unregulated microglia phagocytic activity.

In some embodiments, the agent is selected from any of the chemical moieties (e.g., small molecules) shown in Table 1.

TABLE 1

| | | |
|---|---|---|
| 1 | 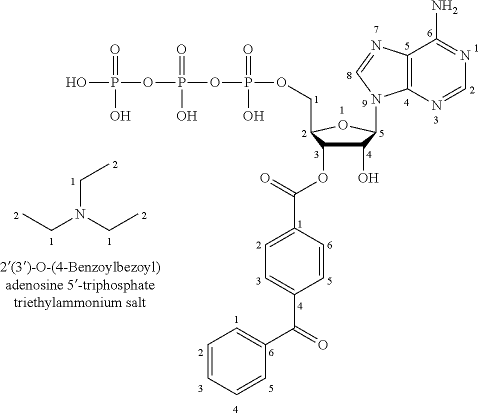 | B6396 |
| | (2R,3S,4R,5R)-5-(6-amino-purin-9-yl)-4-hydroxy-2-(((hydroxy((hydroxy(phosphonooxy) | |
| | phosphoryl)oxy)phosphoryl)oxy)methyl)tetrahydrofuran-3-yl 4-benzoylbenozoate | |
| | (B6396) | |
| 2 | antithymocyte immunoglobulin | DB00098 |
| | (DrugBank Accession Number: DB00098) | |
| 3 | alpha-Linolenic acid: | DB00132 |
| | 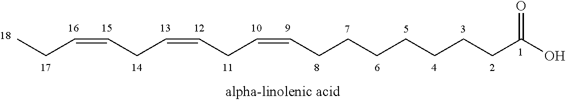 [SMILES: CC/C=C\C/C=C\C/C=C\CCCCCCCC(=O)O] | |
| | (DB00132) | |
| 4 | riboflavin | DB00140 |
| 5 | glutamic acid | DB00142 |
| 6 | glutathione | DB00143 |
| 7 | cysteine | DB00151 |
| 8 | nicotine | DB00184 |
| 9 | cevimeline | DB00185 |
| 10 | troglitazone | DB00197 |
| 11 | caffeine | DB00201 |
| 12 | succinylcholine | DB00202 |
| 13 | enflurane | DB00228 |
| 14 | ranolazine | DB00243 |
| 15 | phenytoin | DB00252 |
| 16 | theophylline | DB00277 |
| 17 | bexarotene | DB00307 |
| 18 | valproic acid | DB00313 |
| 19 | metformin | DB00331 |
| 20 | mefloquine | DB00358 |
| 21 | tacrine | DB00382 |
| 22 | carbamoylcholine | DB00411 |
| 23 | rosiglitazone | DB00412 |
| 24 | spironolactone | DB00421 |
| 25 | levothyroxine | DB00451 |
| 26 | fluoxetine | DB00472 |
| 27 | gallamine triethiodide | DB00483 |
| 28 | dextrothyroxine | DB00509 |
| 29 | lamotrigine | DB00555 |
| 30 | levocarnitine | DB00583 |
| 31 | lisuride | DB00589 |
| 32 | ivermectin | DB00602 |
| 33 | imatinib | DB00619 |
| 34 | adenosine | DB00640 |
| 35 | dyphylline | DB00651 |
| 36 | sumatriptan | DB00669 |
| 37 | galantamine | DB00674 |
| 38 | isoflurophate | DB00677 |
| 39 | lamivudine | DB00709 |
| 40 | paroxetine | DB00715 |
| 41 | riluzole | DB00740 |
| 42 | tretinoin | DB00755 |
| 43 | hexachlorophene | DB00756 |
| 44 | minaprine | DB00805 |
| 45 | pentoxifylline | DB00806 |
| 46 | enprofylline | DB00824 |
| 47 | donepezil | DB00843 |
| 48 | ranitidine | DB00863 |
| 49 | ethanol | DB00898 |
| 50 | methantheline | DB00940 |
| 51 | physostigimine | DB00981 |
| 52 | glyburide | DB01016 |
| 53 | bethanechol | DB01019 |
| 54 | memantine | DB01043 |
| 55 | rifampicin | DB01045 |
| 56 | ibuprofen | DB01050 |
| 57 | melatonin | DB01065 |
| 58 | promethazine | DB01069 |
| 59 | pilocarpine | DB01085 |
| 60 | miconazole | DB01110 |
| 61 | pioglitazone | DB01132 |
| 62 | tiludronic acid | DB01133 |
| 63 | carvedilol | DB01136 |
| 64 | desipramine | DB01151 |
| 65 | halothane | DB01159 |
| 66 | arsenic trioxide | DB01169 |
| 67 | ceftriaxone | DB01212 |
| 68 | aminophylline | DB01223 |
| 69 | metoclopramide | DB01233 |
| 70 | decamethonium | DB01245 |
| 71 | oxtriphylline | DB01303 |
| 72 | ephedrine | DB01364 |
| 73 | rasagiline | DB01367 |
| 74 | mibefradil | DB01388 |
| 75 | bezafibrate | DB01393 |
| 76 | theobromine | DB01412 |
| 77 | zinc | DB01593 |
| 78 | probucol | DB01599 |
| 79 | prasterone | DB01708 |
| 80 | isocitric acid | DB01727 |
| 81 | cordycepin triphosphate | DB01860 |
| 82 | taurine | DB01956 |
| 83 | adenosine-5-diphosphoribose | DB02059 |
| 84 | 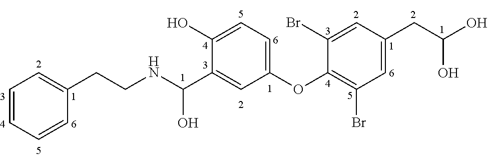 [SMILES: Oc1ccc(Oc2c(Br)cc(CC(O)O)cc2Br)cc1C(O)NCCc1ccccc1] | DB02106 |
| 85 | 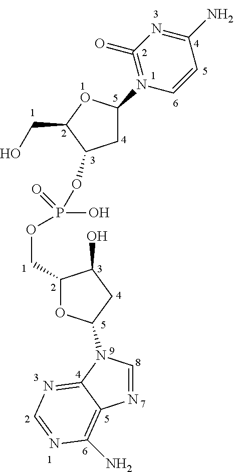 | DB02573 |
| | (2R,3S,5R)-5-(4-amino-2-oxopyrimidin-1(2H)-yl)-2- | |
| | (hydroxymethyl)tetrahydrofuran-3-yl (((2R,3S,5R)-5-(6- | |
| | amino-9H-purin-9-yl)-3-hydroxytetrahydrofuran-2-yl)methyl) | |
| | hydrogen phosphate | |
| 86 | cholic acid | DB02659 |
| 87 | resveratrol | DB02709 |
| 88 | flavin adenine dinucleotide | DB03147 |
| 89 | 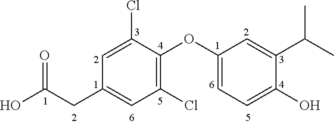 [SMILES: CC(C)c1cc(Oc2c(Cl)cc(CC(=O)O)cc2Cl)ccc1O] | DB03176 |
| | 3,5-dichloro-4-(4-hydroxy-3-isopropylphenoxy)phenylacetic acid | |
| | (KB 141) | |
| 90 | ,5-Dione | DB03181 |
| | (DB03181) | |
| 91 | Flavin mononucleotide | DB03247 |
| 92 | tetrastearoyl cardiolipin | DB03429 |
| 93 | nicotinamide adenine dinucleotide phosphate | DB03461 |
| 94 | naringenin | DB03467 |
| 95 | benzoic acid | DB03793 |
| 96 | thionicotinamide adenine dinucleotide | DB03893 |
| 97 | guanosine-5-triphosphate | DB04137 |
| 98 | quercetin | DB04216 |
| 99 | citric acid | DB04272 |
| 100 | guanosine-5-diphosphate | DB04315 |
| 101 | binodenoson | DB04853 |
| 102 | 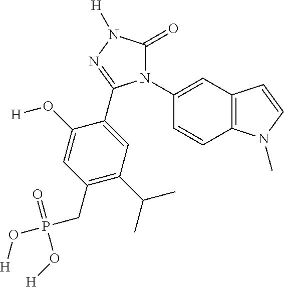 [SMILES: CC(C)c1cc(-c2n[nH]c(=O)n2-c2ccc3c(ccn3C)c2)c(O)cc1CP(=O)(O)O] | DB04932 |
| | (DB04932) | |
| 103 | tamibarotene | DB04942 |
| 104 | apadenoson | DB05009 |
| 105 | eprotirome | DB05035 |
| 106 | 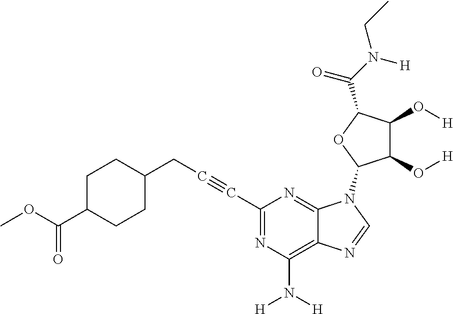 [SMILES: CCNC(=O)[C@H]1O[C@@H](n2cnc3c(N)nc(C#CCC4CCC(C(=O)OC)CC4)nc32)[C@H](O)[C@@H]1O] | DB05191 |
| | (DB05191) | |
| 107 | piclidenoson | DB05511 |
| 108 | CVT-6883 (DB05936) | DB05936 |
| 109 | acetylcysteine | DB06151 |
| 110 | regadenoson | DB06213 |
| 111 | 4-(2-Aminoethyl)Benzenesulfonyl Fluoride (DB07347-2) | DB07347 |
| 112 | sobetirome | DB07425 |
| 113 | epibatidine | DB07720 |
| 114 | | DB08085 |
| 115 | | DB08770 |
| 116 | fingolimod | DB08868 |
| 117 | ponatinib | DB08901 |
| 118 | cannabidiol | DB09061 |
| 119 | copper (DB09130) | DB09130 |
| 120 | doxofylline | DB09273 |
| 121 | aluminum chloride | DB11081 |
| 122 | calcium citrate | DB11093 |
| 123 | pyrantel | DB11156 |
| 124 | calcium phosphate | DB11348 |
| 125 | artenimol | DB11638 |
| 126 | curcumin | DB11672 |
| 127 | istradefylline | DB11757 |
| 128 | brexanolone | DB11859 |
| 129 | fostamatinib | DB12010 |
| 130 | epigallocatechin gallate | DB12116 |
| 131 | sulforaphane | DB12422 |
| | 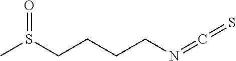 | |
| 132 | enasidenib | DB13874 |
| 133 | 8-chlorotheophylline | DB14132 |
| 134 | calcium phosphate dihydrate | DB14481 |
| 135 | zinc acetate | DB14487 |
| 136 | zinc chloride | DB14533 |
| 137 | 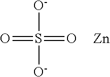 | DB14548 |
| 138 | curcumin sulfate | DB14635 |
| 139 | 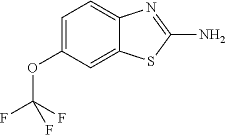 [SMILES: Nc1nc2ccc(OC(F)(F)F)cc2s1] | MolPort-000-151-262 |
| 140 | 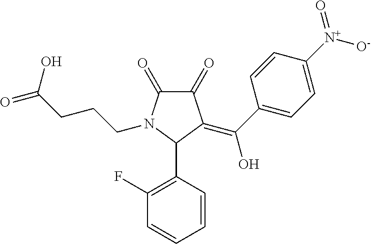 [SMILES: O=C(O)CCCN1C(=O)C(=O)/C(=C(/O)c2ccc([N+](=O)[O-])cc2)C1c1ccccc1F] | MolPort-000-726-476 |
| 141 | 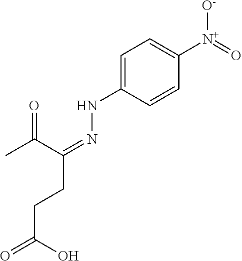 [SMILES: CC(=O)/C(CCC(=O)O)=N\Nc1ccc([N+](=O)[O-])cc1] | MolPort-000-732-885 |
| 142 | 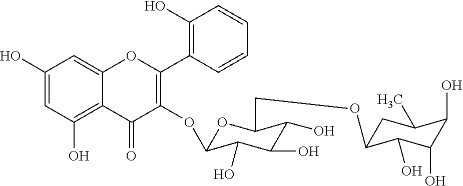 [SMILES: CC1CC(OCC2OC(Oc3c(-c4ccccc4O)oc4cc(O)cc(O)c4c3=O)C(O)C(O)C2O)C(O)C(O)C1O] | MolPort-000-758-142 |
| 143 | 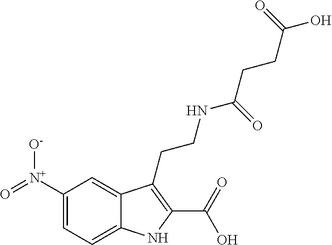 [SMILES: O=C(O)CCC(=O)NCCc1c(C(=O)O)[nH]c2ccc([N+](=O)[O-])cc12] | MolPort-000-823-614 |
| 144 | 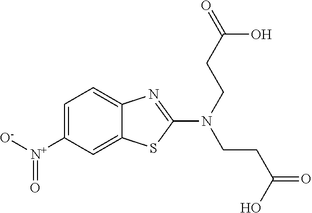 [SMILES: O=C(O)CCN(CCC(=O)O)c1nc2ccc([N+](=O)[O-])cc2s1] | MolPort-001-930-020 |
| 145 | 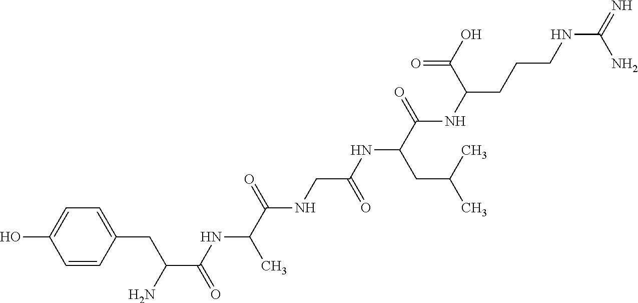 [SMILES: CC(C)CC(NC(=O)CNC(=O)C(C)NC(=O)C(N)Cc1ccc(O)cc1)C(=O)NC(CCCNC(=N)N)C(=O)O] | MolPort-002-136-863 |
| 146 | 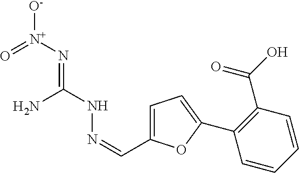 [SMILES: N/C(=N\[N+](=O)[O-])N/N=C\c1ccc(-c2ccccc2C(=O)O)o1] | MolPort-002-147-808 |
| 147 | 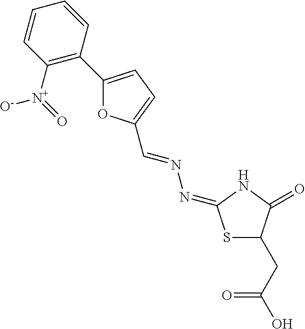 [SMILES: O=C(O)CC1S/C(=N/N=C/c2ccc(-c3ccccc3[N+](=O)[O-])o2)NC1=O] | MolPort-002-216-168 |
| 148 | 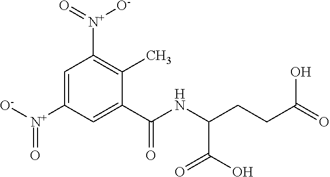 [SMILES: Cc1c(C(=O)NC(CCC(=O)O)C(=O)O)cc([N+](=O)[O-])cc1[N+](=O)[O-]] | MolPort-002-327-349 |
| 149 | 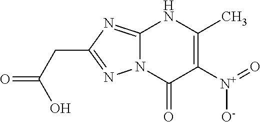 [SMILES: Cc1[nH]c2nc(CC(=O)O)nn2c(=O)c1[N+](=O)[O-]] | MolPort-002-579-160 |
| 150 | 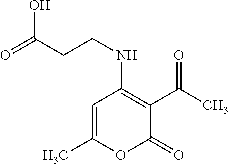 [SMILES: CC(=O)c1c(NCCC(=O)O)cc(C)oc1=O] | MolPort-002-730-310 |
| 151 | 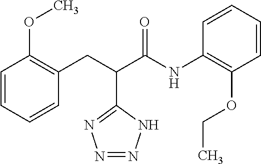 [SMILES: CCOc1ccccc1NC(=O)C(Cc1ccccc1OC)c1nnn[nH]1] | MolPort-002-936-367 |
| 152 | 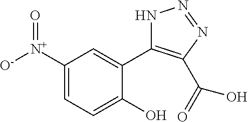 [SMILES: O=C(O)c1nn[nH]c1-c1cc([N+](=O)[O-])ccc1O] | MolPort-002-936-481 |
| 153 | 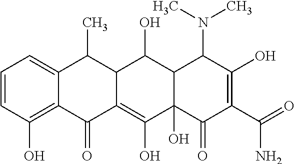 [SMILES: CC1c2cccc(O)c2C(=O)C2=C(O)C3(O)C(=O)C(C(N)=O)=C(O)C(N(C)C)C3C(O)C21] | MolPort-002-964-477 |
| 154 | 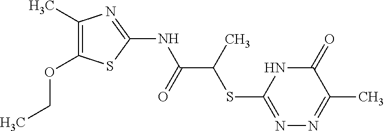 [SMILES: CCOc1sc(NC(=O)C(C)Sc2nnc(C)c(=O)[nH]2)nc1C] | MolPort-002-980-501 |
| 155 | 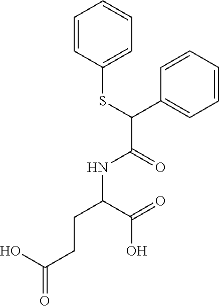 [SMILES: O=C(O)CCC(NC(=O)C(Sc1ccccc1)c1ccccc1)C(=O)O] | MolPort-004-638-720 |
| 156 | 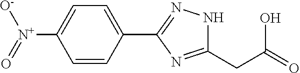 [SMILES: O=C(O)Cc1nc(-c2ccc([N+](=O)[O-])cc2)n[nH]1] | MolPort-004-850-506 |
| 157 | 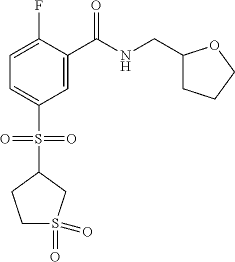 [SMILES: O=C(NCC1CCCO1)c1cc(S(=O)(=O)C2CCS(=O)(=O)C2)ccc1F] | MolPort-008-320-166 |
| 158 | 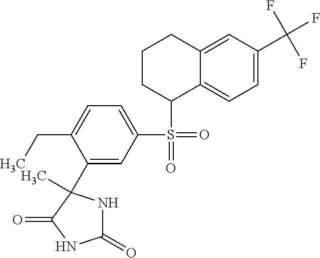 [SMILES: CCc1ccc(S(=O)(=O)C2CCCc3cc(C(F)(F)F)ccc32)cc1C1(C)NC(=O)NC1=O] | MolPort-009-101-544 |
| 159 | 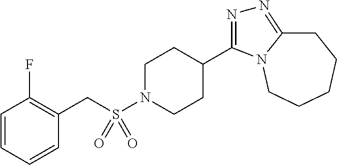 [SMILES: O=S(=O)(Cc1ccccc1F)N1CCC(c2nnc3n2CCCCC3)CC1] | MolPort-016-694-875 |
| 160 | 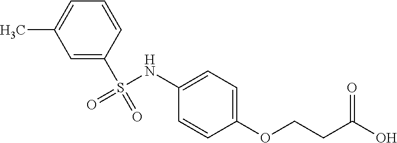 [SMILES: Cc1cccc(S(=O)(=O)Nc2ccc(OCCC(=O)O)cc2)c1] | MolPort-020-093-386 |
| 161 | 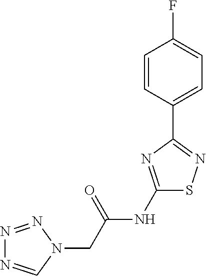 [SMILES: O=C(Cn1cnnn1)Nc1nc(-c2ccc(F)cc2)ns1] | MolPort-023-151-322 |
| 162 | 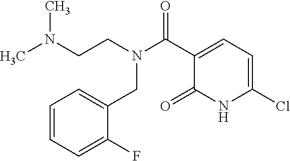 [SMILES: CN(C)CCN(Cc1ccccc1F)C(=O)c1ccc(Cl)[nH]c1=O] | MolPort-023-191-673 |
| 163 | 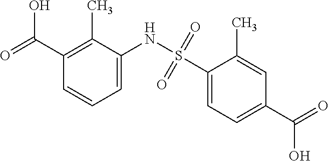 [SMILES: Cc1cc(C(=O)O)ccc1S(=O)(=O)Nc1cccc(C(=O)O)c1C] | MolPort-023-244-339 |
| 164 | 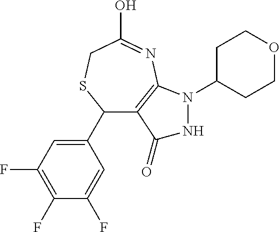 [SMILES: O=c1[nH]n(C2CCOCC2)c2c1C(c1cc(F)c(F)c(F)c1)SCC(O)=N2] | MolPort-030-000-175 |
| 165 | 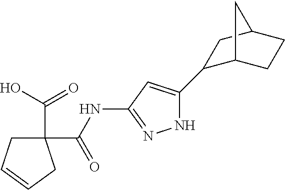 [SMILES: O=C(O)C1(C(=O)Nc2cc(C3CC4CCC3C4)[nH]n2)CC=CC1] | MolPort-046-113-888 |
| 166 | 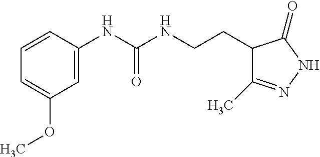 [SMILES: COc1cccc(NC(=O)NCCC2C(=O)NN=C2C)c1] | Z1210573638 |
| 167 | 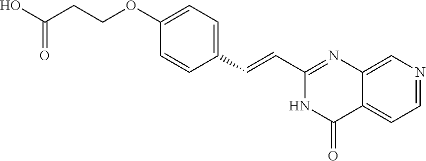 | Z1470517150-6 |
| 168 | 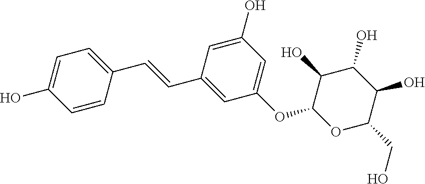 [SMILES: OC[C@@H]1O[C@H](Oc2cc(O)cc(/C=C/c3ccc(O)cc3)c2)[C@@H](O)[C@H](O)[C@H]1O] | ZINC000000899166 |
| 169 | 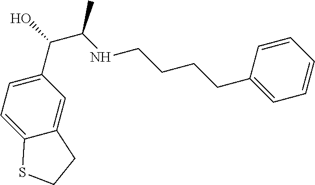 [SMILES: C[C@@H](NCCCCc1ccccc1)[C@@H](O)c1ccc2c(c1)CCS2] | ZINC000001846218 |
| 170 | 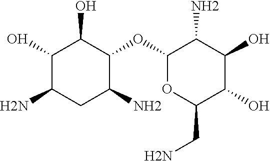 [SMILES: NC[C@H]1O[C@H](O[C@H]2[C@H](O)[C@@H](O)[C@H](N)C[C@@H]2N)[C@H](N)[C@@H](O)[C@@H]1O] | ZINC000004095654 |
| 171 | 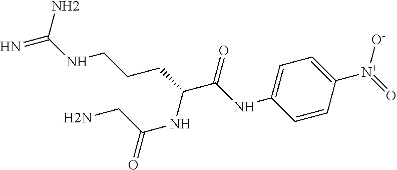 [SMILES: N=C(N)NCCC[C@@H](NC(=O)CN)C(=O)Nc1ccc([N+](=O)[O-])cc1] | ZINC000004545953 |
| 172 | 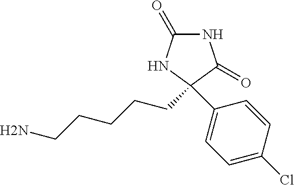 [SMILES: NCCCCC[C@]1(c2ccc(Cl)cc2)NC(=O)NC1=O] | ZINC000006658167 |
| 173 | 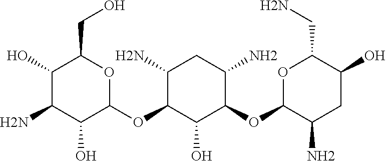 [SMILES: NC[C@H]1O[C@H](O[C@H]2[C@H](O)[C@@H](OC3O[C@H](CO)[C@@H](O)[C@H](N)[C@H]3O)[C@H](N)C[C@@H]2N)[C@H](N)C[C@@H]1O] | ZINC000008214692 |
| 174 | 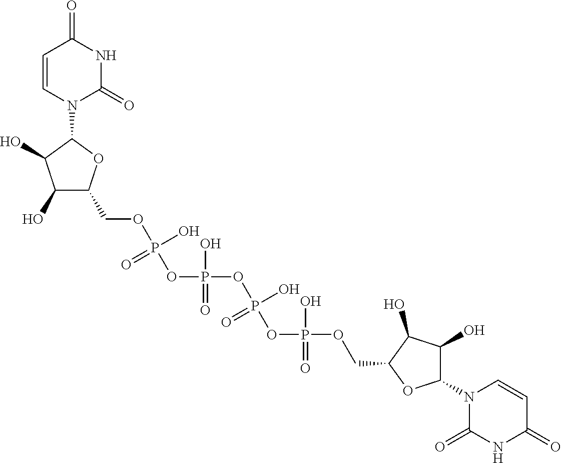 [SMILES: O=c1ccn([C@@H]2O[C@H](COP(=O)(O)OP(=O)(O)OP(=O)(O)OP(=O)(O)OC[C@H]3O[C@@H](n4ccc(=O)[nH]c4=O)[C@H](O)[C@@H]3O)[C@@H](O)[C@H]2O)c(=O)[nH]1] | ZINC000008551963 |
| 175 | 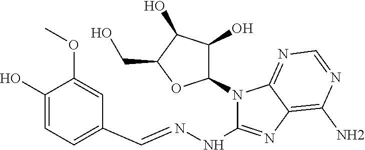 [SMILES: COc1cc(/C=N/Nc2nc3c(N)ncnc3n2[C@H]2O[C@@H](CO)[C@@H](O)[C@H]2O)ccc1O] | ZINC000008740517 |
| 176 | 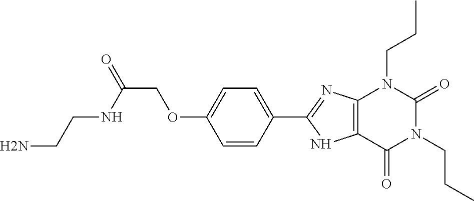 [SMILES: CCCn1c(=O)c2[nH]c(-c3ccc(OCC(=O)NCCN)cc3)nc2n(CCC)c1=O] | ZINC000009210767 |
| 177 | 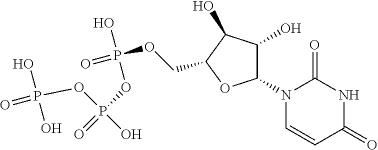 [SMILES: O=c1ccn([C@@H]2O[C@H](CO[P@@](=O)(O)O[P@@](=O)(O)OP(=O)(O)O)[C@@H](O)[C@@H]2O)c(=O)[nH]1] | ZINC000013548378 |
| 178 | 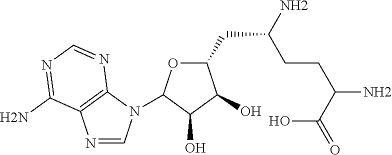 [SMILES: Nc1ncnc2c1ncn2C1O[C@H](C[C@H](N)CCC(N)C(=O)O)[C@@H](O)[C@H]1O] | ZINC000013650200 |
| 179 | 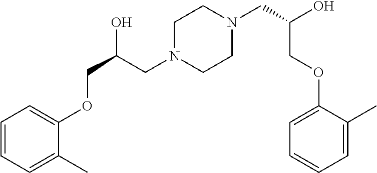 [SMILES: Cc1ccccc1OC[C@@H](O)CN1CCN(C[C@H](O)COc2ccccc2C)CC1] | ZINC000019788892 |
| 180 | 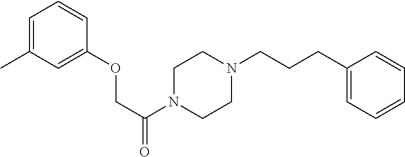 [SMILES: Cc1cccc(OCC(=O)N2CCN(CCCc3ccccc3)CC2)c1] | ZINC000019789335 |
| 181 | 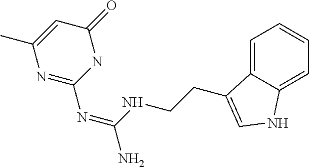 [SMILES: Cc1cc(=O)[nH]c(/N=C(/N)NCCc2c[nH]c3ccccc23)n1] | ZINC000019944488 |
| 182 | 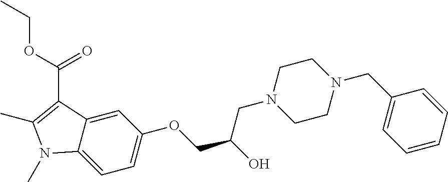 [SMILES: CCOC(=O)c1c(C)n(C)c2ccc(OC[C@H](O)CN3CCN(Cc4ccccc4)CC3)cc12] | ZINC000020060019 |
| 183 | 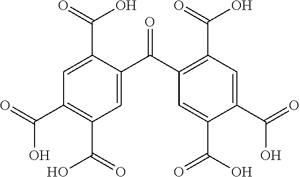 [SMILES: O=C(O)c1cc(C(=O)O)c(C(=O)c2cc(C(=O)O)c(C(=O)O)cc2C(=O)O)cc1C(=O)O] | ZINC000022204540 |
| 184 | 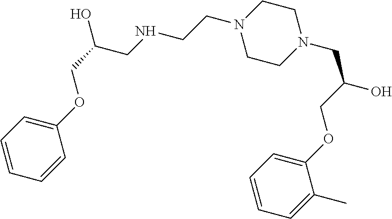 [SMILES: Cc1ccccc1OC[C@H](O)CN1CCN(CCNC[C@@H](O)COc2ccccc2)CC1] | ZINC000022787740 |
| 185 | 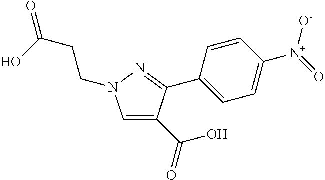 [SMILES: O=C(O)CCn1cc(C(=O)O)c(-c2ccc([N+](=O)[O-])cc2)n1] | ZINC000027642662_88334485 |
| 186 | 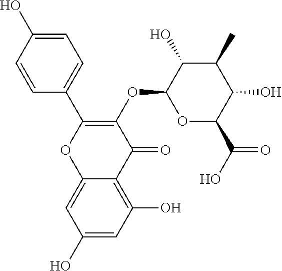 [SMILES: C[C@@H]1[C@@H](O)[C@H](Oc2c(-c3ccc(O)cc3)oc3cc(O)cc(O)c3c2=O)O[C@H](C(=O)O)[C@H]1O] | ZINC000031155995 |
| 187 | 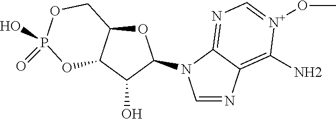 [SMILES: CO[n+]1cnc2c(ncn2[C@@H]2O[C@@H]3CO[P@](=O)(O)O[C@H]3[C@H]2O)c1N] | ZINC000038580931 |
| 188 | 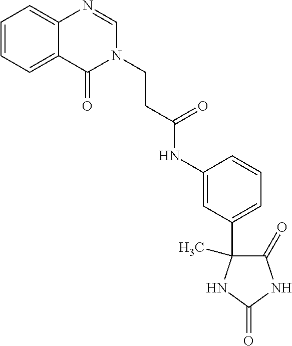 [SMILES: CC1(c2cccc(NC(=O)CCn3cnc4ccccc4c3=O)c2)NC(=O)NC1=O] | ZINC000040110952 |
| 189 | 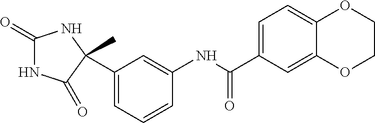 [SMILES: C[C@]1(c2cccc(NC(=O)c3ccc4c(c3)OCCO4)c2)NC(=O)NC1=O] | ZINC000040111044 |
| 190 | 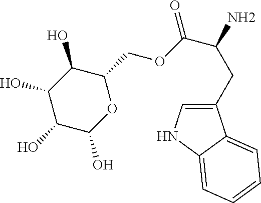 [SMILES: N[C@@H](Cc1c[nH]c2ccccc12)C(=O)OC[C@@H]1O[C@H](O)[C@H](O)[C@H](O)[C@H]1O] | ZINC000061989702 |
| 191 | 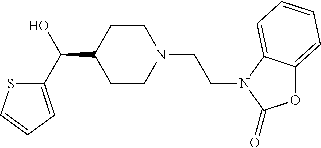 [SMILES: O=c1oc2ccccc2n1CCN1CCC([C@H](O)c2cccs2)CC1] | ZINC000067642267 |
| 192 | 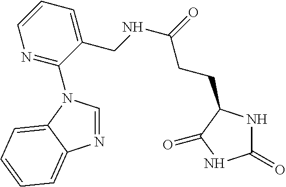 [SMILES: O=C(CC[C@H]1NC(=O)NC1=O)NCc1cccnc1-n1cnc2ccccc21] | ZINC000067675558 |
| 193 | 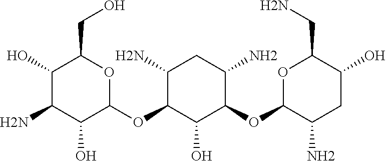 [SMILES: NC[C@@H]1O[C@H](O[C@H]2[C@H](O)[C@@H](OC3O[C@H](CO)[C@@H](O)[C@H](N)[C@H]3O)[C@H](N)C[C@@H]2N)[C@@H](N)C[C@H]1O] | ZINC000070680696 |
| 194 | 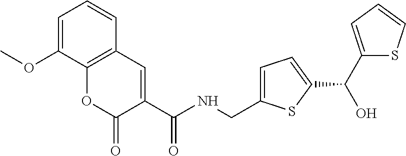 [SMILES: COc1cccc2cc(C(=O)NCc3ccc([C@@H](O)c4cccs4)s3)c(=O)oc12] | ZINC000090613649 |
| 195 | 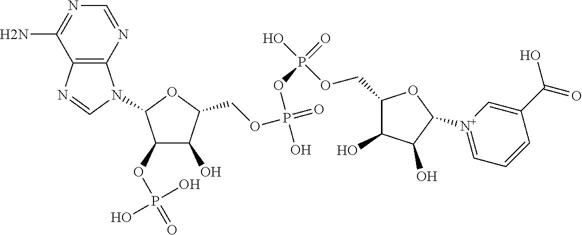 [SMILES: Nc1ncnc2c1ncn2[C@@H]1O[C@H](COP(=O)(O)O[P@](=O)(O)OC[C@@H]2O[C@H]([n+]3cccc(C(=O)O)c3)[C@@H](O)[C@H]2O)[C@@H](O)[C@H]1OP(=O)(O)O] | ZINC000095543597 |
| 196 | 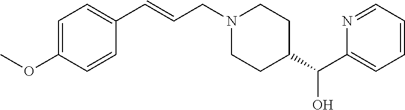 [SMILES: COc1ccc(/C=C/CN2CCC([C@@H](O)c3ccccn3)CC2)cc1] | ZINC000097759359 |
| 197 | 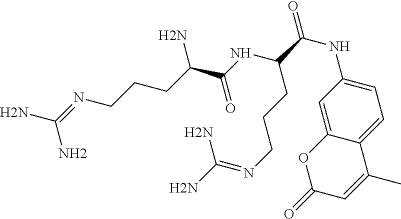 | ZINC000098043870 |
| 198 | 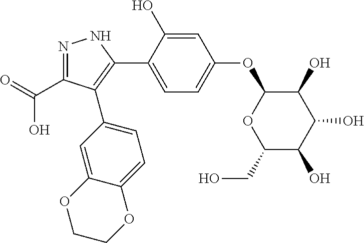 [SMILES: O=C(O)c1n[nH]c(-c2ccc(O[C@@H]3O[C@@H](CO)[C@H](O)[C@@H](O)[C@@H]3O)cc2O)c1-c1ccc2c(c1)OCCO2] | ZINC000253497753 |
| 199 | 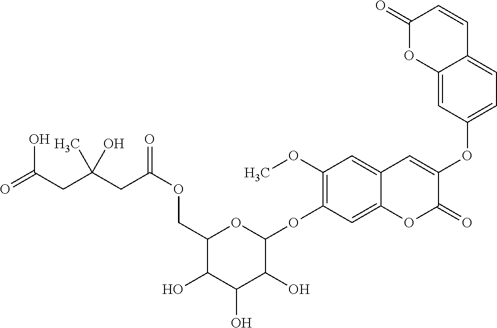 [SMILES: COc1cc2cc(Oc3ccc4ccc(=O)oc4c3)c(=O)oc2cc1OC1OC(COC(=O)CC(C)(O)CC(=O)O)C(O)C(O)C1O] | ZINC000253501136 |
| 200 | 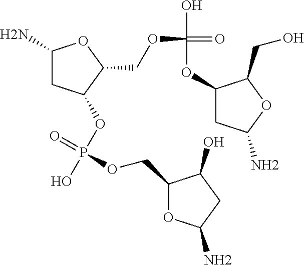 | ZINC000255963400 |
| 201 | 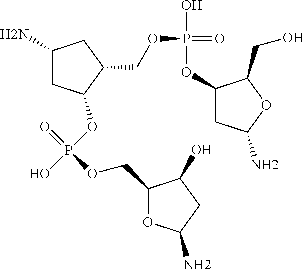 [SMILES: N[C@@H]1C[C@H](CO[P@](=O)(O)O[C@@H]2C[C@@H](N)O[C@@H]2CO)[C@H](O[P@@](=O)(O)OC[C@@H]2O[C@H](N)C[C@@H]2O)C1] | ZINC000263583759 |
| 202 | 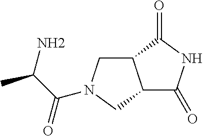 | ZINC000387198271 |
| 203 | 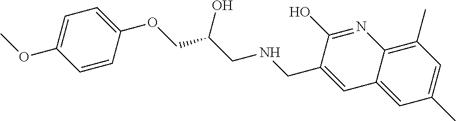 | ZINC000585284939 |
| 204 | 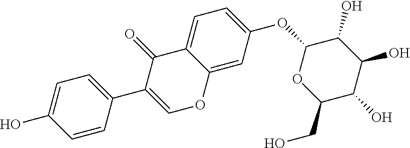 [SMILES: O=c1c(-c2ccc(O)cc2)coc2cc(O[C@H]3O[C@H](CO)[C@@H](O)[C@H](O)[C@H]3O)ccc12] | ZINC13548856 |
| 205 | 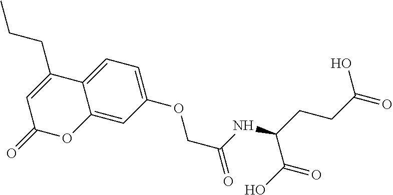 [SMILES: CCCc1cc(=O)oc2cc(OCC(=O)N[C@@H](CCC(=O)O)C(=O)O)ccc12] | ZINC2120846 |
| 206 | 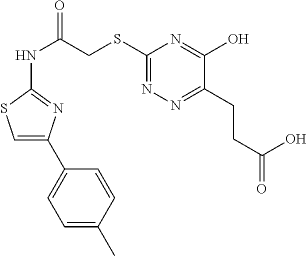 [SMILES: Cc1ccc(-c2csc(NC(=O)CSc3nnc(CCC(=O)O)c(O)n3)n2)cc1] | ZINC22799470 |
| 207 | 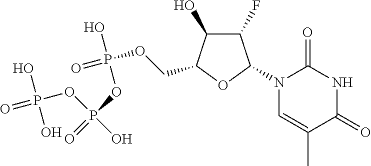 [SMILES: Cc1cn([C@@H]2O[C@H](CO[P@](=O)(O)O[P@](=O)(O)OP(=O)(O)O)[C@@H](O)[C@@H]2F)c(=O)[nH]c1=O] | ZINC31458084 |
| 208 | 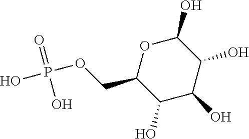 [SMILES: O=P(O)(O)OC[C@H]1O[C@@H](O)[C@H](O)[C@@H](O)[C@@H]1O] | ZINC3875374 |
| 209 | 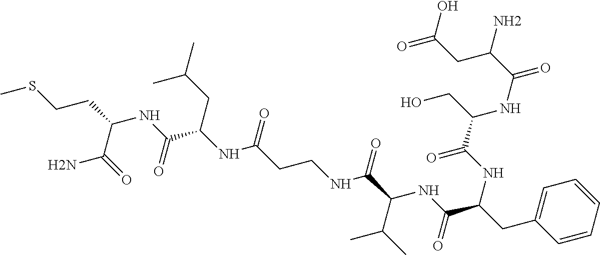 [SMILES: CSCC[C@H](NC(=O)[C@H](CC(C)C)NC(=O)CCNC(=O)[C@@H](NC(=O)[C@H](Cc1ccccc1)NC(=O)[C@H](CO)NC(=O)C(N)CC(=O)O)C(C)C)C(N)=O] | ZINC95543647 |
| 210 | Allyl isothiocyanate | Allyl_isothiocyanate_500.png |
| 211 | Allylglucosinolate (sinigrin) | Allylglucosinolate_500.png |
| 212 | 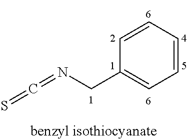 | Benzyl_isothiocyanate_500.png |
| 213 | Benzylglucosinolate | Benzylglucosinolate_500.png |
| | 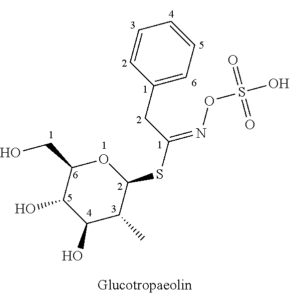 [SMILES: C[C@@H]1[C@@H](O)[C@H](O)[C@@H](CO)O[C@H]1S/C(Cc1ccccc1)=N/OS(=O)(=O)O] | |
| 214 | 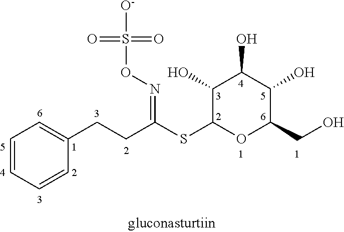 [SMILES: O=S(=O)([O-])O/N=C(\CCc1ccccc1)SC1O[C@H](CO)[C@@H](O)[C@H](O)[C@H]1O] | Gluconasturtiin_500.png |
| | ((E)-3-phenyl-1-(((3R,4S,5S,6R)-3,4,5-trihydroxy-6-(hydroxymethyl)tetrahydro-2H-pyran-2- | |
| | yl)thio)propylidene)amino sulfate | |
| 215 | 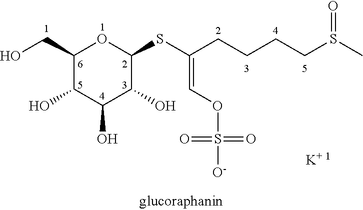 [SMILES: CS(=O)CCCC/C(=C\OS(=O)(=O)[O-])S[C@@H]1O[C@H](CO)[C@@H](O)[C@H](O)[C@H]1O] | Glucoraphanin_500.png |
| | ((E)-5-(methylsulfinyl)-1-(((2S,3R,4S,5S,6R)-3,4,5-trihydroxy-6-(hydroxymethyl)tetrahydro-2H- | |
| | pyran-2-yl)thio)pentylidene)amino sulfate | |
| 216 | 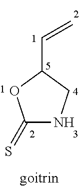 | Goitrin_500.png |
| 217 | 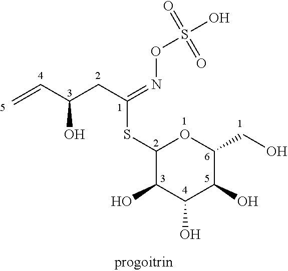 [SMILES: C=C[C@H](O)C/C(=N\OS(=O)(=O)O)SC1O[C@H](CO)[C@@H](O)[C@H](O)[C@H]1O] | Progoitrin_500.png |

In some embodiments, the agent is selected from phenyl isothiocyanate (PEITC), an analog of PEITC, oxidized nicotinamide adenine dinucleotide (NAD+), and reduced nicotinamide adenine dinucleotide (NADH).

In some embodiments, a PEITC analog is any chemical moiety related to watercress and/or other cruciferous plant extraction and the structures related to PEITC.

In some embodiments, the PEITC analog is selected from

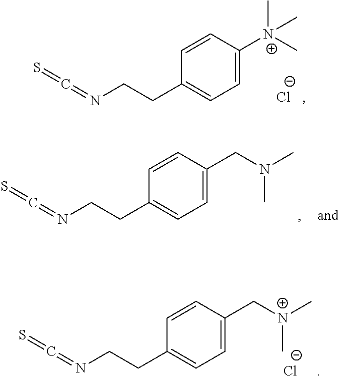

In some embodiments, the PEITC analog is selected from:

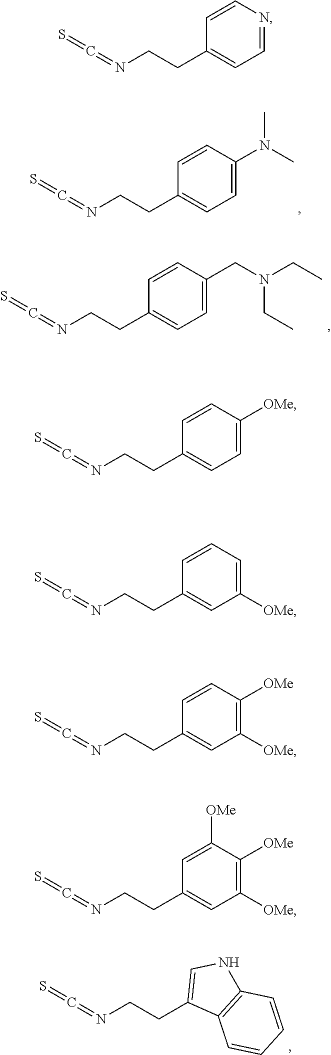

Allylglucosinolate (sinigrin), allyl isothiocyanate, Benzylglucosinolate (Glucotropaeolin), benzyl isothiocyanate, Gluconasturtiin, (R)-4-(methylsulfinyl)butylglucosinolate (Glucoraphanin), (R)-4-(methylsulfinyl)butyl isothiocyanate (sulforaphane), (R)-2-hydroxybut-3-enylglucosinolate (progoitrin), and (S)-5-vinyloxazolidine-2-thione (goitrin).

In certain embodiments, the one or more agents capable of protecting neurons from cell death and unregulated microglia phagocytic activity may be comprised within any type or kind of composition. For example, in some embodiments, such a composition may be an over-the-counter composition, a pharmaceutical composition, or any kind of cosmetic composition.

In certain embodiments, the present provides the following compounds:

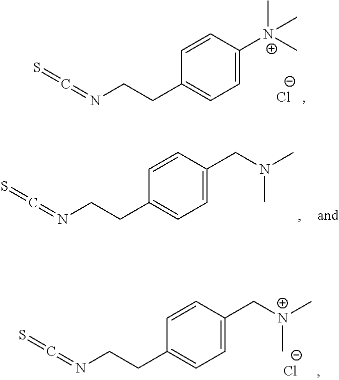

including pharmaceutically acceptable salts, solvates, and/or prodrugs thereof.

In certain embodiments, the present invention provides a composition comprising one or more (e.g., 1, 2, 3, 4, 5, 6, 7, 8, 9, 10, 15, 20, 25, 50, 100, 200, etc.) of the following: one or more of the chemical moieties (e.g., small molecule) shown in Table 1, PEITC, an analog of PEITC, NAD+, and NADH.

In some embodiments, a PEITC analog is any chemical moiety related to watercress and/or other cruciferous plant extraction and the structures related to PEITC.

In some embodiments, the PEITC analog is selected from: N

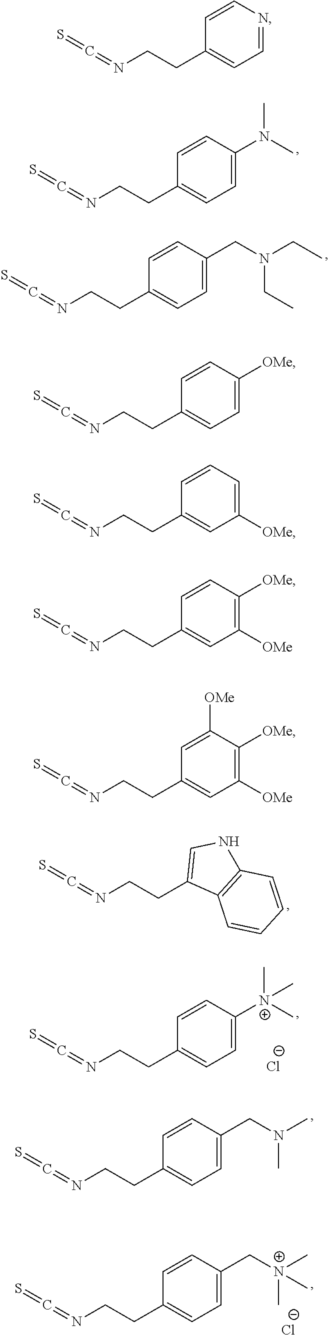

Allylglucosinolate (sinigrin), allyl isothiocyanate, Benzylglucosinolate (Glucotropaeolin), benzyl isothiocyanate, Gluconasturtiin, (R)-4-(methylsulfinyl)butylglucosinolate (Glucoraphanin), (R)-4-(methylsulfinyl)butyl isothiocyanate (sulforaphane), (R)-2-hydroxybut-3-enylglucosinolate (progoitrin), and (S)-5-vinyloxazolidine-2-thione (goitrin).

In some embodiments, the composition is an over-the-counter composition, or a pharmacological prescription.

In certain embodiments, the present invention provides an over-the-counter composition comprising one or more (e.g., 1, 2, 3, 4, 5, 6, 7, 8, 9, 10, 15, 20, 25, 50, 100, 200, etc.) of the following: one or more of the chemical moieties (e.g., small molecule) shown in Table 1, PEITC, an analog of PEITC, NAD+, and NADH.

In some embodiments, a PEITC analog is any chemical moiety related to watercress and/or other cruciferous plant extraction and the structures related to PEITC.

In some embodiments, the PEITC analog is selected from:

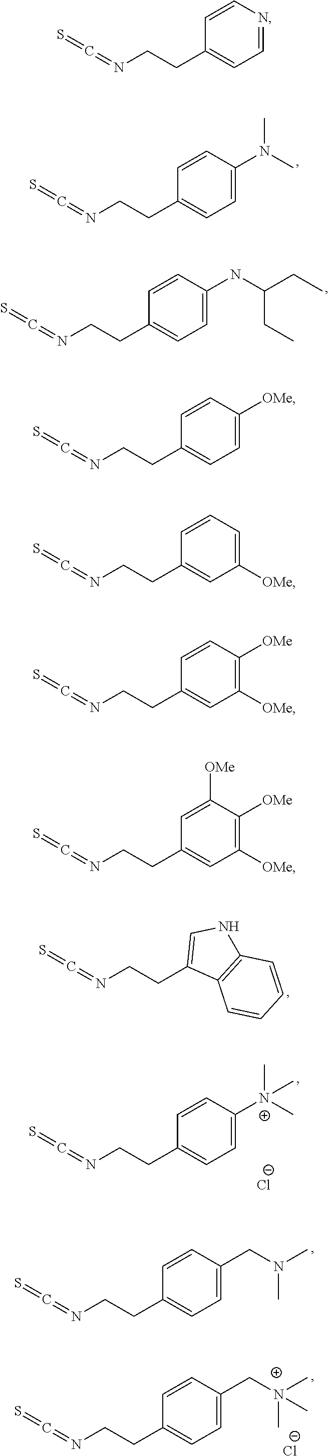

Allylglucosinolate (sinigrin), allyl isothiocyanate, Benzylglucosinolate (Glucotropaeolin), benzyl isothiocyanate, Gluconasturtiin, (R)-4-(methylsulfinyl)butylglucosinolate (Glucoraphanin), (R)-4-(methylsulfinyl)butyl isothiocyanate (sulforaphane), (R)-2-hydroxybut-3-enylglucosinolate (progoitrin), and (S)-5-vinyloxazolidine-2-thione (goitrin). In some embodiments, the over-the-counter composition is a tablet, capsule, powder, suspension, or solution.

### Definitions

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising/* or “having” certain elements are also contemplated as “consisting essentially of and “consisting of those certain elements.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “over-the-counter” means to provide by retail purchase without a prescription or license from a physician or medical practitioner (e.g., does not require a prescription from a physician in order to be administered to the human).

As used herein, the term “pharmaceutical compound” refers to any physical state of a material. Pharmaceutical compounds include but are not limited to capsules, tablets, liquids, topical formulations, and inhaled formulations.

Unless otherwise defined, all technical terms used herein have the same meaning as commonly understood by one of ordinary skill in the art to which this disclosure belongs.

### BRIEF DESCRIPTION OF THE DRAWINGS

FIG. 1 shows neuron cell viability percentage upon exposure to various compounds.

FIG. 2 shows microglia cell viability percentage upon exposure to various compounds.

### DETAILED DESCRIPTION

AD is a complex neurodegenerative disease that involves systemic pathological changes. These changes include the accumulation of amyloid beta (Aβ) peptide into senile plaques around neurons, and the formation of tau protein “tangles” inside neurons. While these two factors are the most well-known and studied aspects of AD pathology, many other perturbations in multiple pathways contribute to the development and progression of the disease.

The present invention relates generally to neurodegenerative diseases and conditions (e.g., Alzheimer's disease) characterized with dysfunctional energetic function, unregulated microglia phagocytic activity and other related de-regulated biological functions. This invention further relates to methods and compositions for treating such neurodegenerative diseases and conditions with pharmaceutical compositions comprising one or more agents capable of protecting neurons from cell death and unregulated microglia phagocytic activity.

In certain embodiments, the present invention provides a method of treating a mammal suffering from a neurodegenerative disorder comprising administering to the mammal a composition comprising one or more agents capable of protecting neurons from cell death and unregulated microglia phagocytic activity.

In certain embodiments, the present invention provides a method for preventing and/or inhibiting neuronal cell death in a mammal in need thereof, the method comprising administering to the mammal a composition comprising one or more agents capable of protecting neurons from cell death and unregulated microglia phagocytic activity.

In certain embodiments, the present invention provides a method for preventing and/or inhibiting unregulated microglia phagocytic activity in a mammal in need thereof, the method comprising administering to the mammal a composition comprising one or more agents capable of protecting neurons from cell death and unregulated microglia phagocytic activity.

In some embodiments, the neurodegenerative disorder is selected from AD, Parkinson's disease, Huntington's disease, amyotrophic lateral sclerosis, motor neuron disease, subjective memory complaints, and MCI. In some embodiments, the AD is an early stage, prodromal phase of AD or late stage.

In some embodiments, the mammal is a human patient.

In certain embodiments, the present invention provides a method for preventing and/or inhibiting neuronal cell death in a subject suffering from a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) comprising, consisting of, or consisting essentially of administering to the subject a therapeutically effective amount of one or more agents capable of preventing and/or inhibiting unregulated microglia phagocytic activity.

In certain embodiments, the present invention provides for preventing and/or inhibiting unrelated microglia phagocytic activity in neuronal cells of a subject suffering from a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) comprising, consisting of, or consisting essentially of administering to the subject a therapeutically effective amount of one or more agents capable of preventing and/or inhibiting unregulated microglia phagocytic activity.

In certain embodiments, the present invention provides a method of preventing the onset of a neurodegenerative disorder (e.g., AD (e.g., early stage, prodromal phase, late stage), Parkinson's disease, Huntington's disease, amyotrophic lateral sclerosis, motor neuron disease, subjective memory complaints, and MCI) in a subject (e.g., a human subject) comprising, consisting of, or consisting essentially of administering to the subject a therapeutically effective amount of one or more agents capable of preventing and/or inhibiting unregulated microglia phagocytic activity.

In certain embodiments, the present invention provides a method of treating and/or ameliorating the symptoms of a neurodegenerative disorder (e.g., AD (e.g., early stage, prodromal phase, late stage), Parkinson's disease, Huntington's disease, amyotrophic lateral sclerosis motor neuron disease, subjective memory complaints, and MCI) in a subject (e.g., a human subject) comprising, consisting of, or consisting essentially of administering to the subject a therapeutically effective amount of one or more agents capable of preventing and/or inhibiting unregulated microglia phagocytic activity.

Such methods are not limited to use of a particular agent capable of protecting neurons from cell death and unregulated microglia phagocytic activity.

In some embodiments, the agent is selected from any of the chemical moieties (e.g., small molecules) shown in Table 1.

In some embodiments, the agent is selected from phenyl isothiocyanate (PEITC), an analog of PEITC, oxidized nicotinamide adenine dinucleotide (NAD+), and reduced nicotinamide adenine dinucleotide (NADH).

In some embodiments, a PEITC analog is any chemical moiety related to watercress and/or other cruciferous plant extraction and the structures related to PEITC.

In some embodiments, the PEITC analog is selected from

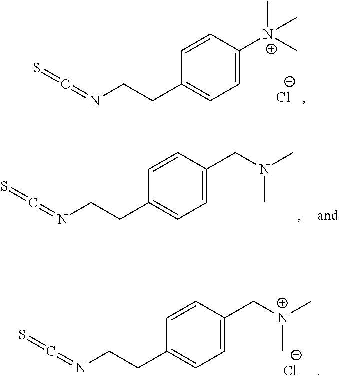

In some embodiments, the PEITC analog is selected from:

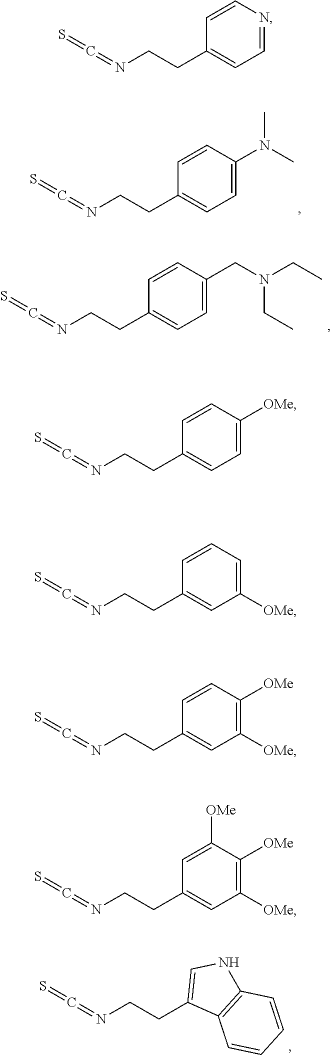

Allylglucosinolate (sinigrin), allyl isothiocyanate, Benzylglucosinolate (Glucotropaeolin), benzyl isothiocyanate, Gluconasturtiin, (R)-4-(methylsulfinyl)butylglucosinolate (Glucoraphanin), (R)-4-(methylsulfinyl)butyl isothiocyanate (sulforaphane), (R)-2-hydroxybut-3-enylglucosinolate (progoitrin), and (S)-5-vinyloxazolidine-2-thione (goitrin).

In certain embodiments, the one or more agents capable of protecting neurons from cell death and unregulated microglia phagocytic activity may be comprised within any type or kind of composition. For example, in some embodiments, such a composition may be an over-the-counter composition, a pharmaceutical composition, or any kind of cosmetic composition.

In certain embodiments, the present provides the following compounds:

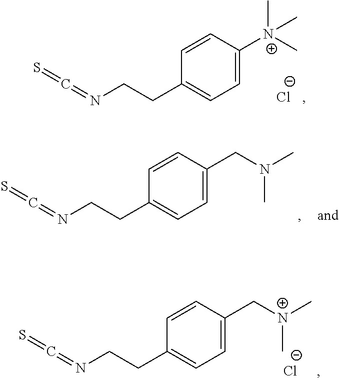

including pharmaceutically acceptable salts, solvates, and/or prodrugs thereof.

In certain embodiments, the present invention provides a composition comprising one or more (e.g., 1, 2, 3, 4, 5, 6, 7, 8, 9, 10, 15, 20, 25, 50, 100, 200, etc.) of the following: one or more of the chemical moieties (e.g., small molecule) shown in Table 1, PEITC, an analog of PEITC, NAD+, and NADH.

In some embodiments, a PEITC analog is any chemical moiety related to watercress and/or other cruciferous plant extraction and the structures related to PEITC.

In some embodiments, the PEITC analog is selected from:

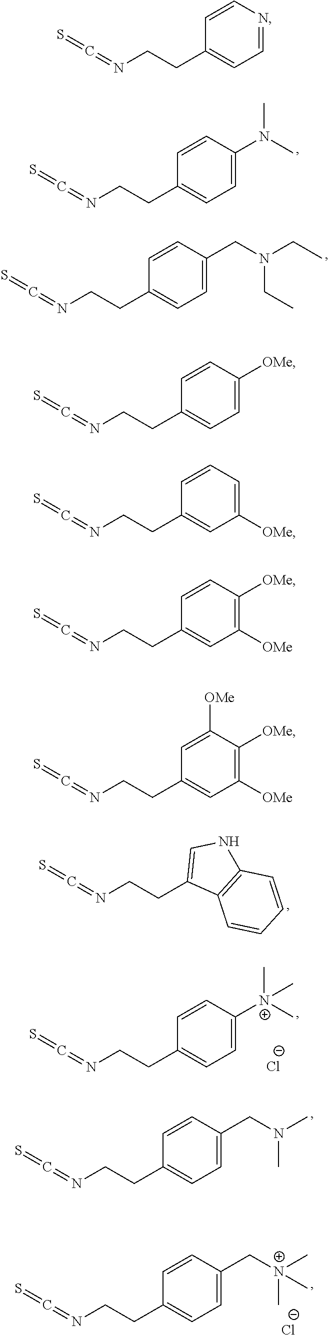

Allylglucosinolate (sinigrin), allyl isothiocyanate, Benzylglucosinolate (Glucotropaeolin), benzyl isothiocyanate, Gluconasturtiin, (R)-4-(methylsulfinyl)butylglucosinolate (Glucoraphanin), (R)-4-(methylsulfinyl)butyl isothiocyanate (sulforaphane), (R)-2-hydroxybut-3-enylglucosinolate (progoitrin), and (S)-5-vinyloxazolidine-2-thione (goitrin).

In some embodiments, the composition is an over-the-counter composition, or a pharmacological prescription.

In certain embodiments, the present invention provides an over-the-counter composition comprising one or more (e.g., 1, 2, 3, 4, 5, 6, 7, 8, 9, 10, 15, 20, 25, 50, 100, 200, etc.) of the following: one or more of the chemical moieties (e.g., small molecule) shown in Table 1, PEITC, an analog of PEITC, NAD+, and NADH.

In some embodiments, a PEITC analog is any chemical moiety related to watercress and/or other cruciferous plant extraction and the structures related to PEITC.

In some embodiments, the PEITC analog is selected from:

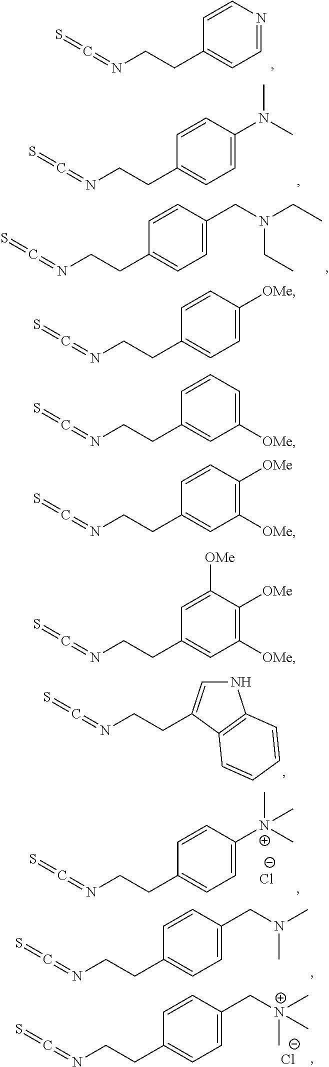

Allylglucosinolate (sinigrin), allyl isothiocyanate, Benzylglucosinolate (Glucotropaeolin), benzyl isothiocyanate, Gluconasturtiin, (R)-4-(methylsulfinyl)butylglucosinolate (Glucoraphanin), (R)-4-(methylsulfinyl)butyl isothiocyanate (sulforaphane), (R)-2-hydroxybut-3-enylglucosinolate (progoitrin), and (S)-5-vinyloxazolidine-2-thione (goitrin). In some embodiments, the over-the-counter composition is a tablet, capsule, powder, suspension, or solution.

The methods and compositions of the present invention are useful in treating mammals. Such mammals include humans as well as non-human mammals. Non-human mammals include, for example, companion animals such as dogs and cats, agricultural animals such live stock including cows, horses and the like, and exotic animals, such as zoo animals.

Treatment can include administration of an effective amount of one or more of an agent capable of protecting neurons from cell death and unregulated microglia phagocytic activity.

Administration can be by any suitable route of administration including buccal, dental, endocervical, intramuscular, inhalation, intracranial, intralymphatic, intramuscular, intraocular, intraperitoneal, intrapleural, intrathecal, intratracheal, intrauterine, intravascular, intravenous, intravesical, intranasal, ophthalmic, oral, otic, biliary perfusion, cardiac perfusion, priodontal, rectal, spinal subcutaneous, sublingual, topical, intravaginal, transermal, ureteral, or urethral. Dosage forms can be aerosol including metered aerosol, chewable bar, capsule, capsule containing coated pellets, capsule containing delayed release pellets, capsule containing extended release pellets, concentrate, cream, augmented cream, suppository cream, disc, dressing, elixer, emulsion, enema, extended release fiber, extended release film, gas, gel, metered gel, granule, delayed release granule, effervescent granule, chewing gum, implant, inhalant, injectable, injectable lipid complex, injectable liposomes, insert, extended release insert, intrauterine device, jelly, liquid, extended release liquid, lotion, augmented lotion, shampoo lotion, oil, ointment, augmented ointment, paste, pastille, pellet, powder, extended release powder, metered powder, ring, shampoo, soap solution, solution for slush, solution/drops, concentrate solution, gel forming solution/drops, sponge, spray, metered spray, suppository, suspension, suspension/drops, extended release suspension, swab, syrup, tablet, chewable tablet, tablet containing coated particles, delayed release tablet, dispersible tablet, effervescent tablet, extended release tablet, orally disintegrating tablet, tampon, tape or troche/lozenge.

Intraocular administration can include administration by injection including intravitreal injection, by eyedrops and by trans-scleral delivery.

Administration can also be by inclusion in the diet of the mammal such as in a functional food for humans or companion animals.

It is also contemplated that certain formulations containing compositions comprising one or more agents capable of protecting neurons from cell death and unregulated microglia phagocytic activity are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methylcellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated such as to provide rapid, sustained, or delayed release of the active ingredients after administration to the patient by employing procedures well known in the art. The formulations can also contain substances that diminish proteolytic degradation and promote absorption such as, for example, surface-active agents.

The specific dose can be calculated according to the approximate body weight or body surface area of the patient or the volume of body space to be occupied. The dose will also depend upon the particular route of administration selected. Further refinement of the calculations necessary to determine the appropriate dosage for treatment is routinely made by those of ordinary skill in the art. Such calculations can be made without undue experimentation by one skilled in the art in light of the activity in assay preparations such as has been described elsewhere for certain compounds (see for example, Howitz et al., Nature 425:191-196, 2003 and supplementary information that accompanies the paper). Exact dosages can be determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

The present invention also provides kits comprising one or more of agents capable of protecting neurons from cell death and unregulated microglia phagocytic activity and instructions for administering the agent to an animal (e.g., a human patient suffering from a neurodegenerative disorder (e.g., AD)). The kits may optionally contain other therapeutic agents.

### EXPERIMENTAL

The following examples are provided to demonstrate and further illustrate certain preferred embodiments of the present invention and are not to be construed as limiting the scope thereof. As used herein, the use of pronouns (e.g., “our”, “we”, etc.) refers to the inventors.

### Example I

### High-Throughput Screening Methods

### Cell Viability Assay

SH-SY5Y human neuroblastoma cell line was differentiated into human neurons by using the protocol adapting from a previous paper (Shipley et al, 2016). Cells were seeded at the 96 well plate for differentiation into neurons for 18 days and continued culturing for another 18 days. The screening for our compounds for differentiated neurons were performed with the utilization of robotic liquid handling system (BiomekFX). At day 1 of the screening, 1 uM human Aβ (1-42) were added to fresh medium and cells. The cells were then incubated for 24 hours. After 24 hours, the compounds were prepared at the concentration of 10 mM and would be diluted to the final concentration at 10 uM for the screening. The cells were incubated for 24 hours. At day 3 of the screening, 4 hours ahead, 20 ul of MTT were put to each well containing 200 ul media. After 4 hours of incubation in the presence of MTT reagent, 200 ul of DMSO (Sigma-Aldrich) was added to each well to solubilize formazan crystals. The cell viability was determined by the colorimetric signal was quantified by measurement of optical density (λ=570 nm) using an ultraviolet/visual spectrophotometric plate reader (Clariostar).

### Aβ Uptake Assay

Before the screening, primary human microglia cells were obtained from Celprogen (NC1632783) and seeded at the 96 well plate. With the utilization of robotic liquid handling system (BiomekFX), 1 uM fluorescently labeled human Aβ (1-42) were added to fresh medium and cells. After 24 hours of incubation, the compounds were prepared at the concentration of 10 mM and would be diluted to the final concentration at 10 uM for the screening. The cells were incubated for 24 hours and then Aβ (1-42) uptake in primary cultures of human microglia was measured by removal of the medium containing fluorescent Ab(1-42) and addition of 200 ul of ice-cold PBS to stop cellular uptake mechanisms. At this time, cells were solubilized with 100 ul of 0.2% sodium dodecyl sulfate for 30 minutes. The human Aβ (1-42) signal was measured using a fluorescent assay plate reader (Clariostar) at an excitation wavelength of 450 nm and an emission wavelength of 535 nm.

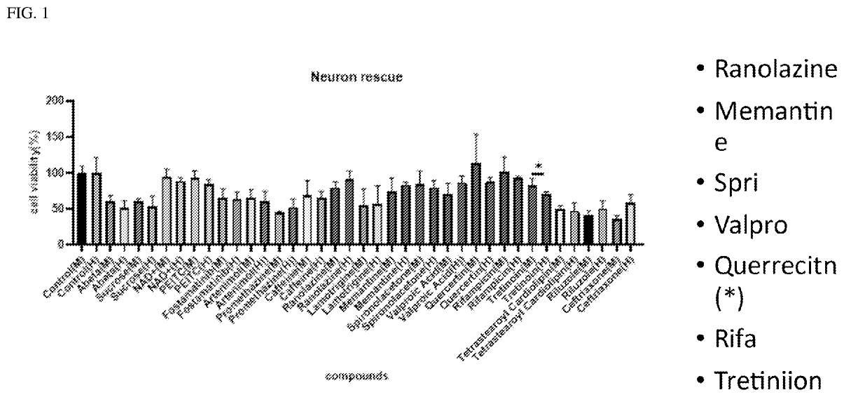

FIG. 1 shows neuron cell viability percentage upon exposure to various compounds.

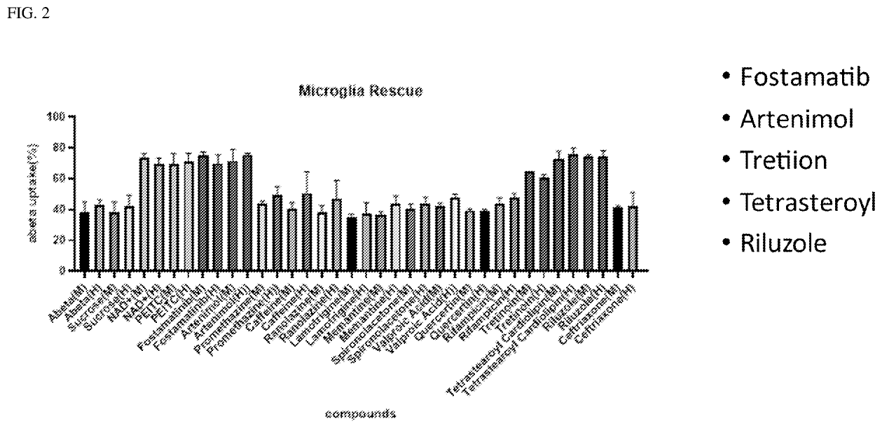

FIG. 2 shows microglia cell viability percentage upon exposure to various compounds.

### Example II

This example describes the synthesis of PEITC analogs (see scheme 1).

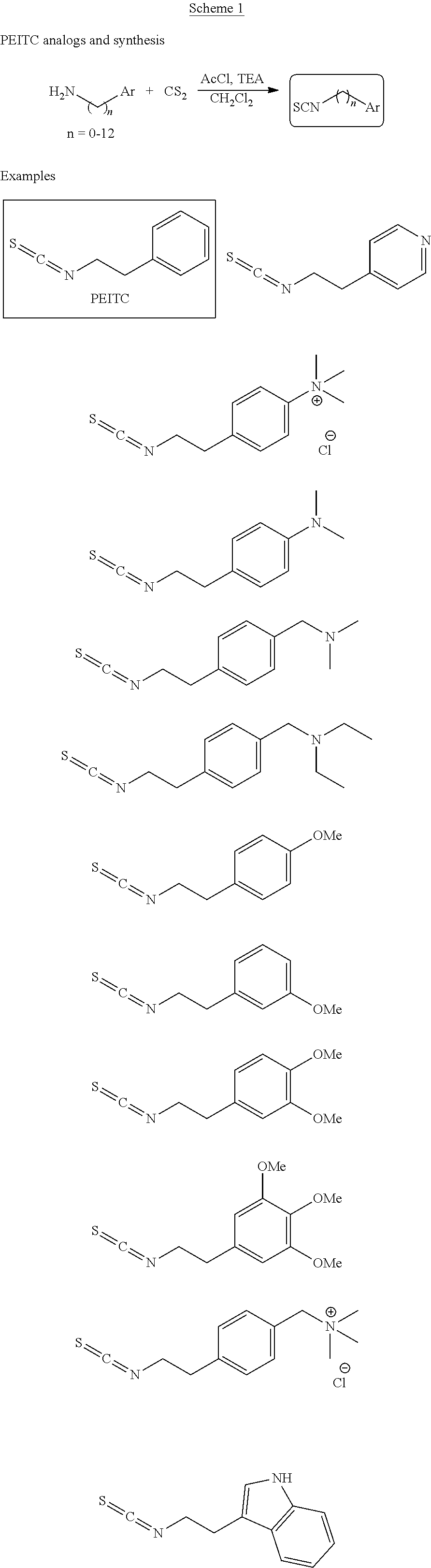

The structures contain isothiocynate and aromatic ring connected by (CH₂)n (n=0-12). Aromatic refers to: phenyl and fused phenyl ring and the rings tethering substituent groups such as alkyl, aryl, Cl, Br, F, I, amino, OH, alkoxy, etc and heteroaromatics such as pyridine, indole, quinoline etc and these heteroaromatics attaching substituent groups such as alkyl, aryl, Cl, Br, F, I, amino, OH, alkoxy, etc. Some selected examples are provided shown below. We have purchased and/or made some of these compounds for biological activities. They can be prepared by reaction of corresponding amines with CS₂ in the presence of acetyl chloride (AcCl) and TEA (triethyl amine).

Additional experiments will be conducted with these PEITC analogs using high-throughout screening with MTT and Abeta uptake assays to assess the therapeutic efficacy of such PEITC analogs in the treatment of neurodegenerative disorders as described herein.

Additional in vivo experiments will be conducted with these PEITC analogs to assess the therapeutic efficacy of such PEITC analogs in the treatment of neurodegenerative disorders as described herein.

### INCORPORATION BY REFERENCE

The entire disclosure of each of the patent documents and scientific articles referred to herein is incorporated by reference for all purposes.

The following references denoted throughout the application are incorporated by references in their entireties:
Kim, T., C. S. Lim, and B. K. Kaang, Cell type-specific gene expression profiling in brain tissue: comparison between TRAP, LCM and RNA-seq. BMB Rep, 2015. 48(7): p. 388-94.
Zhao, Y., et al., TREM2 Is a Receptor for beta-Amyloid that Mediates Microglial Function. Neuron, 2018. 97(5): p. 1023-1031 e7.
Rajendran, L. and R. C. Paolicelli, Microglia-Mediated Synapse Loss in Alzheimer's Disease. J Neurosci, 2018. 38(12): p. 2911-2919.
Condello, C., P. Yuan, and J. Grutzendler, Microglia-Mediated Neuroprotection, TREM2, and Alzheimer's Disease: Evidence From Optical Imaging. Biol Psychiatry, 2018. 83(4): p. 377-387.
Sims, R., et al., Rare coding variants in PLCG2, ABI3, and TREM2 implicate microglial-mediated innate immunity in Alzheimer's disease. Nat Genet, 2017. 49(9): p. 1373-1384.
Zeisel, A., et al., Brain structure. Cell types in the mouse cortex and hippocampus revealed by single-cell RNA-seq. Science, 2015. 347(6226): p. 1138-42.
Zhang, Y., et al., An RNA-sequencing transcriptome and splicing database of glia, neurons, and vascular cells of the cerebral cortex. J Neurosci, 2014. 34(36): p. 11929-47.
Rui Chang, M. A., Kwangsik Won, Gabi Kastermuelle, Andrew Sarkin, Rima Kaddurah-Daouk. Predictive Network Modeling identifies Sex-specific Metabolic Drivers in Alzheimer's Disease. in Alzheimer's & Dementia (In Submission). 2018.
Canevelli, M., et al., Sex and gender differences in the treatment of Alzheimer's disease: A systematic review of randomized controlled trials. Pharmacol Res, 2017. 115: p. 218-223.
Zhao, L., et al., Sex differences in metabolic aging of the brain: insights into female susceptibility to Alzheimer's disease. Neurobiol Aging, 2016. 42: p. 69-79.
Ronquillo, J. G., M. R. Baer, and W. T. Lester, Sex-specific patterns and differences in dementia and Alzheimer's disease using informatics approaches. J Women Aging, 2016. 28(5): p. 403-11.
Mazure, C. M. and J. Swendsen, Sex differences in Alzheimer's disease and other dementias. Lancet Neurol, 2016. 15(5): p. 451-2.
Laws, K. R., K. Irvine, and T. M. Gale, Sex differences in cognitive impairment in Alzheimer's disease. World J Psychiatry, 2016. 6(1): p. 54-65.
Munro, C. A., Sex differences in Alzheimer's disease risk: are we looking at the wrong hormones? Int Psychogeriatr, 2014. 26(10): p. 1579-84.
Zhang, B., et al., Integrated systems approach identifies genetic nodes and networks in late-onset Alzheimer's disease. Cell, 2013. 153(3): p. 707-20.
Chang, R., R. Shoemaker, and W. Wang, Systematic search for recipes to generate induced pluripotent stem cells. PLoS Comput Biol, 2011. 7(12): p. e1002300.
Chang, R., R. Shoemaker, and W. Wang, A novel knowledge-driven systems biology approach for phenotype prediction upon genetic intervention. IEEE/ACM Trans Comput Biol Bioinform, 2011. 8(5): p. 1170-82.
Chang, R., J. R. Karr, and E. E. Schadt, Causal inference in biology networks with integrated belief propagation. Pac Symp Biocomput, 2015: p. 359-70.
Carcamo-Orive, I., et al., Analysis of Transcriptional Variability in a Large Human iPSC Library Reveals Genetic and Non-genetic Determinants of Heterogeneity. Cell Stem Cell, 2017. 20(4): p. 518-532.
Zissimopoulos, J. M., et al., Sex and Race Differences in the Association Between Statin Use and the Incidence of Alzheimer Disease. JAMA Neurol, 2017. 74(2): p. 225-232.
Chen, Y. Q., et al., Variations in DNA elucidate molecular networks that cause disease. Nature, 2008. 452(7186): p. 429-435.
Schadt, E. E., Molecular networks as sensors and drivers of common human diseases. Nature, 2009. 461(7261): p. 218-223.
Schadt, E. E. and J. L. Bjorkegren, NEW: network-enabled wisdom in biology, medicine, and health care. Sci Transl Med, 2012. 4(115): p. 115rv1.
Gandy, S., et al., CR1 and the “vanishing amyloid” hypothesis of Alzheimer's disease. Biol Psychiatry, 2013. 73(5): p. 393-5.
Rui Chang* #, K. Z., Kruti Patel, Marc R Henrion, Sara Moein, Min Tang, Philip De Jager, Elizabeth Bradshaw, Predictive Network Analysis Identified Novel Therapeutic Targets in Microglials for Alzheimer's diseases. Nature Genetics (In Submission), 2018.
Ivan Carcamo-Orive, M. Y. R. H., Kuixi Zhu, Paige Cundiff, Noam D. Beckmann, Sunita D'Souza, Eric E. Schadt, Thomas Quertermous, Joshua W. Knowles, Rui Chang, Predictive network modeling in human induced pluripotent stem cells identifies key driver genes for insulin responsiveness. Nature Genetics (In Submission), 2018.
Rui Chang, W. W. Novel algorithm for Bayesian network parameter learning with informative prior constraints. in Neural Networks (IJCNN), The 2010 International Joint Conference on. 2010.
Toledo, J. B., et al., Metabolic Network Failures in Alzheimer Disease—A Biochemical Roadmap. Alzheimer's & Dementia, 2017.
Cohain, A., et al., Exploring the Reproducibility of Probabilistic Causal Molecular Network Models. Pac Symp Biocomput, 2016. 22: p. 120-131.
Myers, A., et al., THE HUMAN BRAINOME: HUMAN BRAIN GENOME, TRANSCRIPTOME, AND PROTEOME INTEGRATION, in Alzheimer's & Dementia: The Journal of the Alzheimer's Association. 2016. p. 237-238.
Zhao, X., et al., Resolving complex structural genomic rearrangements using a randomized approach. Genome Biol, 2016. 17(1): p. 126.
Chen, X., et al., Manta: rapid detection of structural variants and indels for germline and cancer sequencing applications. Bioinformatics, 2016. 32(8): p. 1220-2.
Layer, R. M., et al., LUMPY: a probabilistic framework for structural variant discovery. Genome Biol, 2014. 15(6): p. R84.
Pinto, D., et al., Comprehensive assessment of array-based platforms and calling algorithms for detection of copy number variants. Nat Biotechnol, 2011. 29(6): p. 512-20.
Colella, S., et al., QuantiSNP: an Objective Bayes Hidden-Markov Model to detect and accurately map copy number variation using SNP genotyping data. Nucleic Acids Res, 2007. 35(6): p. 2013-25.
Wang, K., et al., PennCNV: an integrated hidden Markov model designed for high-resolution copy number variation detection in whole-genome SNP genotyping data. Genome Res, 2007. 17(11): p. 1665-74.
Darmanis, S., et al., A survey of human brain transcriptome diversity at the single cell level. Proc Natl Acad Sci USA, 2015. 112(23): p. 7285-90.
Carrillo-de Sauvage, M. A., et al., Potent and multiple regulatory actions of microglial glucocorticoid receptors during CNS inflammation. Cell Death Differ, 2013. 20(11): p. 1546-57.
Nadra, K., et al., Phosphatidic acid mediates demyelination in Lpin1 mutant mice. Genes Dev, 2008. 22(12): p. 1647-61.
Bana, L., et al., Liposomes bi-functionalized with phosphatidic acid and an ApoE-derived peptide affect Abeta aggregation features and cross the blood-brain-barrier: implications for therapy of Alzheimer disease. Nanomedicine, 2014. 10(7): p. 1583-90.
Sivendran, S., et al., Dissection of immune gene networks in primary melanoma tumors critical for antitumor surveillance of patients with stage II-III resectable disease. J Invest Dermatol, 2014. 134(8): p. 2202-11.
Xie, Z. R. and M. J. Hwang, Ligand-binding site prediction using ligand-interacting and binding site-enriched protein triangles. Bioinformatics, 2012. 28(12): p. 1579-85.
Xie, Z. R. and M. J. Hwang, Methods for predicting protein-ligand binding sites. Methods Mol Biol, 2015. 1215: p. 383-98.
Xie, Z. R., et al., LISE: a server using ligand-interacting and site-enriched protein triangles for prediction of ligand-binding sites. Nucleic Acids Res, 2013. 41(Web Server issue): p. W292-6.
Luco, J. M., Prediction of the brain-blood distribution of a large set of drugs from structurally derived descriptors using partial least-squares (PLS) modeling. J Chem Inf Comput Sci, 1999. 39(2): p. 396-404.
Subramanian, A., et al., A Next Generation Connectivity Map: L1000 Platform and the First 1,000,000 Profiles. Cell, 2017. 171(6): p. 1437-1452 e17.
Eto, K., et al., Role of NADH shuttle system in glucose-induced activation of mitochondrial metabolism and insulin secretion. Science, 1999. 283(5404): p. 981-5.
Stein, L. R. and S. Imai, The dynamic regulation of NAD metabolism in mitochondria. Trends Endocrinol Metab, 2012. 23(9): p. 420-8.
Pearl, S. M., et al., Effects of NADH on dopamine release in rat striatum. Synapse, 2000. 36(2): p. 95-101.
Vrecko, K., et al., NADH stimulates endogenous dopamine biosynthesis by enhancing the recycling of tetrahydrobiopterin in rat phaeochromocytoma cells. Biochim Biophys Acta, 1997. 1361(1): p. 59-65.
Birkmayer, J. G., et al., Nicotinamide adenine dinucleotide (NADH)—a new therapeutic approach to Parkinson's disease. Comparison of oral and parenteral application. Acta Neurol Scand Suppl, 1993. 146: p. 32-5.
Forsyth, L. M., et al., Therapeutic effects of oral NADH on the symptoms of patients with chronic fatigue syndrome. Ann Allergy Asthma Immunol, 1999. 82(2): p. 185-91.
Birkmayer, J. G., Coenzyme nicotinamide adenine dinucleotide: new therapeutic approach for improving dementia of the Alzheimer type. Ann Clin Lab Sci, 1996. 26(1): p. 1-9.
Demarin, V., et al., Treatment of Alzheimer's disease with stabilized oral nicotinamide adenine dinucleotide: a randomized, double-blind study. Drugs Exp Clin Res, 2004. 30(1): p. 27-33.
Ghareghani, M., et al., Fluvoxamine stimulates oligodendrogenesis of cultured neural stem cells and attenuates inflammation and demyelination in an animal model of multiple sclerosis. Sci Rep, 2017. 7(1): p. 4923.
Ryan, K. J., et al., A human microglia-like cellular model for assessing the effects of neurodegenerative disease gene variants. Sci Transl Med, 2017. 9(421).
Elyaman, W., et al., BAD and Bcl-2 regulation are early events linking neuronal endoplasmic reticulum stress to mitochondria-mediated apoptosis. Brain Res Mol Brain Res, 2002. 109(1-2): p. 233-8.
Elyaman, W., C. Yardin, and J. Hugon, Involvement of glycogen synthase kinase-3beta and tau phosphorylation in neuronal Golgi disassembly. J Neurochem, 2002. 81(4): p. 870-80.
Maia, L. F., et al., Changes in amyloid-beta and Tau in the cerebrospinal fluid of transgenic mice overexpressing amyloid precursor protein. Sci Transl Med, 2013. 5(194): p. 194re2.
Radde, R., et al., Abeta42-driven cerebral amyloidosis in transgenic mice reveals early and robust pathology. EMBO Rep, 2006. 7(9): p. 940-6.
Chang, R., Zhu Kuixi, Arnold Matthias, Kwangsik Nho, Henrion Marc, Moein Sara, Blach Collete, Tenenbaum J D, Kastenmueller G, Saykin A J, Eugenia T, Brinton Roberta, Kaddurah-Daouk Raduk, Predictive Network Analysis Identified Sex, Genotype and Clinic-stage specific Metabolite Fuels for Alzheimer's disease. Nature Metabolism (In Submission), 2018.
Bush, E. C., et al., PLATE-Seq for genome-wide regulatory network analysis of high-throughput screens. Nat Commun, 2017. 8(1): p. 105.
Harold, D., et al., Genome-wide association study identifies variants at CLU and PICALM associated with Alzheimer's disease. Nat Genet, 2009. 41(10): p. 1088-93.
Lambert, J. C., et al., Genome-wide association study identifies variants at CLU and CR1 associated with Alzheimer's disease. Nat Genet, 2009. 41(10): p. 1094-9.
Bu, G., Apolipoprotein E and its receptors in Alzheimer's disease: pathways, pathogenesis and therapy. Nat Rev Neurosci, 2009. 10(5): p. 333-44.
Kanekiyo, T., H. Xu, and G. Bu, ApoE and Abeta in Alzheimer's disease: accidental encounters or partners? Neuron, 2014. 81(4): p. 740-54.
Castellano, J. M., et al., Human apoE isoforms differentially regulate brain amyloid-beta peptide clearance. Sci Transl Med, 2011. 3(89): p. 89ra57.
Sakae, N., et al., ABCA7 Deficiency Accelerates Amyloid-beta Generation and Alzheimer's Neuronal Pathology. J Neurosci, 2016. 36(13): p. 3848-59.
Shinohara, M., et al., APOE2 eases cognitive decline during aging: clinical and preclinical evaluations. Ann Neurol, 2016.
Liu, C. C., et al., Deficiency in LRP6-mediated Wnt signaling contributes to synaptic abnormalities and amyloid pathology in Alzheimer's disease. Neuron, 2014. 84(1): p. 63-77.
Dieterle, F., et al., Renal biomarker qualification submission: a dialog between the FDA-EMEA and Predictive Safety Testing Consortium. Nat Biotechnol, 2010. 28(5): p. 455-62.
Tachibana, M., et al., Rescuing effects of RXR agonist bexarotene on aging-related synapse loss depend on neuronal LRP1. Exp Neurol, 2016. 277: p. 1-9.
Cunnane, S. C. et al. Brain energy rescue: an emerging therapeutic concept for neurodegenerative disorders of ageing. Nature Reviews Drug Discovery (2020).
Collaborators GBDD. Global, regional, and national burden of Alzheimer's disease and other dementias, 1990-2016: a systematic analysis for the Global Burden of Disease Study 2016. Lancet Neurol. 18, 88-106 (2019).
Naseri, N. N. et al. The complexity of tau in Alzheimer's disease. Neuroscience Letters 705, 183-194 (2019).
Lane, C. A., Hardy, J., & Schott, J. M. Alzheimer's disease. Eur J Neurol. 25, 59-70 (2018).
Jaeger, L. B. et al. Testing the neurovascular hypothesis of Alzheimer's disease: LRP-1 antisense reduces blood-brain barrier clearance, increases brain levels of amyloid-beta protein, and impairs cognition. J Alzheimers Dis. 17, 553-70 (2009).
Ronaldson, P. T., & Davis, T. P. Regulation of blood-brain barrier integrity by microglia in health and disease: A therapeutic opportunity. (forthcoming).
Akhmetzyanova, E., Kletenkov, K., Mukhamedshina, Y., & Rizvanov, A. Different Approaches to Modulation of Microglia Phenotypes After Spinal Cord Injury. Front Syst Neurosci. 13, 37 (2019).
Bard, F. et al. Peripherally administered antibodies against amyloid beta-peptide enter the central nervous system and reduce pathology in a mouse model of Alzheimer disease. Nature Medicine 6, 916-919 (2000).
Salloway, S. et al. Two phase 3 trials of Bapineuzumab in mild-to-moderate Alzheimer's disease. The New England Journal of Medicine 370, 322-333 (2014).
Vandenberghe, R. et al. Bapineuzumab for mild to moderate Alzheimer's disease in two global, randomized, phase 3 trials. Alzheimer's Research & Therapy 8 (2016).
Doody, R. et al. A Phase 3 Trial of Semagacestat for Treatment of Alzheimer's Disease. The New England Journal of Medicine 369, 341-350 (2013).
Timmers, M. et al. Pharmacodynamics of atabecestat (JNJ-54861911), an oral BACE1 inhibitor in patients with early Alzheimer's disease: randomized, double-blind, placebo-controlled study. Alzheimer's Research & Therapy 10, 85 (2018).
Novak, G. et al. Long-term safety and tolerability of atabecestat (JNJ-54861911), an oral BACE1 inhibitor, in early Alzheimer's disease spectrum patients: a randomized, double-blind, placebo-controlled study and a two-period extension study. Alzheimer's Research & Therapy 12, 58 (2020).
Kennedy, M. E. et al. The BACE1 inhibitor verubecestat (MK-8931) reduces CNS (3-amyloid in animal models and in Alzheimer's disease patients. Science Translational Medicine 8, 363ra150 (2016).
Egan, M. F. et al. Randomized trial of verubecestat for prodromal Alzheimer's disease. The New England Journal of Medicine 380, 1408-1420 (2019a).
Egan, M. F. et al. Further analyses of the safety of verubecestat in the phase 3 EPOCH trial of mild-to-moderate Alzheimer's disease. Alzheimer's Research & Therapy 11, 68 (2019b).
Petyuk, V. A. et al. The human brainome: network analysis identifies HSPA2 as a novel Alzheimer's disease target. Brain 141, 2721-2739 (2018).
Patel, K. R. et al. Single Cell-type Integrative Network Modeling Identified Novel Microglial-specific Targets for the Phagosome in Alzheimer's Disease. Preprint at https://www.biorxiv.org/content/10.1101/2020.06.09.143529v1 (2020).
Petyuk, V. A. et al. The human brainome: network analysis identifies HSPA2 as a novel Alzheimer's disease target. Brain 141, 2721-2739 (2018).

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating a mammal suffering from a neurodegenerative disorder comprising administering to the mammal a composition comprising one or more agents capable of protecting neurons from cell death and unregulated microglia phagocytic activity.

2. The method of claim 1, wherein the composition is capable of protecting neurons from cell death and unregulated microglia phagocytic activity.

3. The method of claim 1, wherein the neurodegenerative disorder is selected from AD, Parkinson's disease, Huntington's disease, amyotrophic lateral sclerosis, motor neuron disease, subjective memory complaints, and MCI.

4. The method of claim 3, wherein the AD is an early stage, prodromal phase of AD.

5. The method of claim 1, wherein the mammal is a human patient.

6. The method of claim 1, wherein the one or more agents capable of protecting neurons from cell death and unregulated microglia phagocytic activity are selected from the chemical moieties (e.g., small molecules) shown in Table 1.

7. The method of claim 1, wherein the one or more agents capable of protecting neurons from cell death and unregulated microglia phagocytic activity are selected from: PEITC, an analog of PEITC, NADH, NAD+.

8. The method of claim 7, wherein the PEITC analog is selected from the group consisting of: 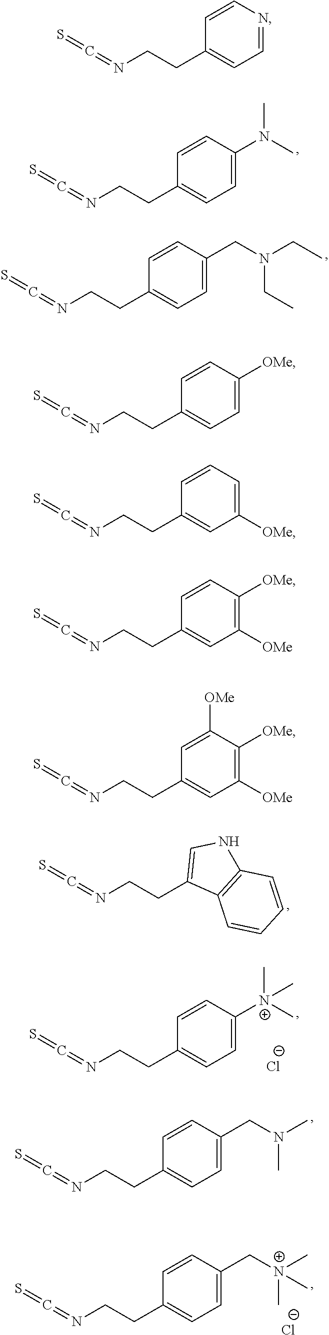
Allylglucosinolate (sinigrin), allyl isothiocyanate, Benzylglucosinolate (Glucotropaeolin), benzyl isothiocyanate, Gluconasturtiin, (R)-4-(methylsulfinyl)butylglucosinolate (Glucoraphanin), (R)-4-(methylsulfinyl)butyl isothiocyanate (sulforaphane), (R)-2-hydroxybut-3-enylglucosinolate (progoitrin), (S)-5-vinyloxazolidine-2-thione (goitrin), and any chemical moiety related to watercress and/or other cruciferous plant extraction.

9-50. (canceled)

51. A composition comprising one or more of the following: one or more of the chemical moieties (e.g., small molecules) shown in Table 1, PEITC, an analog of PEITC, NAD+, and NADH.

52. The composition of claim 51, wherein the PEITC analog is selected from: 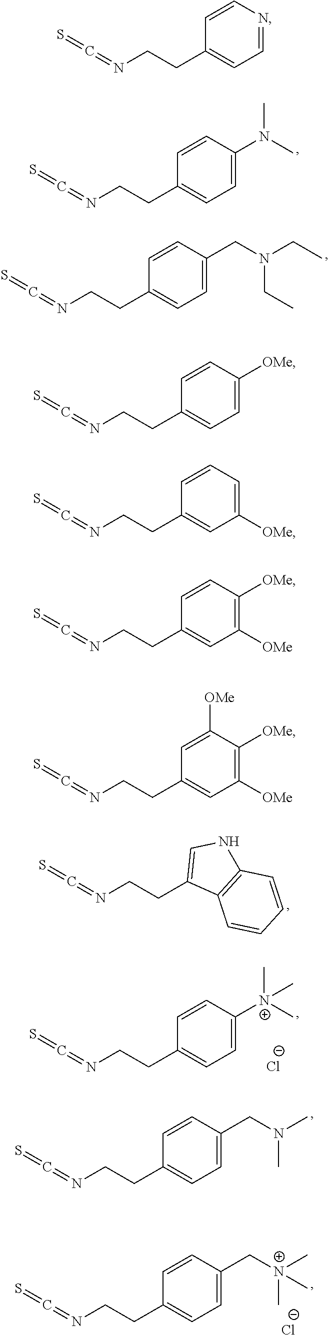
Allylglucosinolate (sinigrin), allyl isothiocyanate, Benzylglucosinolate (Glucotropaeolin), benzyl isothiocyanate, Gluconasturtiin, (R)-4-(methylsulfinyl)butylglucosinolate (Glucoraphanin), (R)-4-(methylsulfinyl)butyl isothiocyanate (sulforaphane), (R)-2-hydroxybut-3-enylglucosinolate (progoitrin), (S)-5-vinyloxazolidine-2-thione (goitrin), and any chemical moiety related to watercress and/or other cruciferous plant extraction.

53. The composition of claim 51, wherein the composition is an over-the-counter composition, or a pharmacological prescription.

54-56. (canceled)
